(19) United States
(12) Patent Application Publication
(10) Pub. No.: US 2019/0082654 A1
(43) Pub. Date: Mar. 21, 2019

(54) SYSTEMS, METHODS AND APPARATUS FOR MONITORING ANIMAL HEALTH CONDITIONS

(71) Applicant: David S. Robbins, Garden City, KS (US)

(72) Inventor: David S. Robbins, Garden City, KS (US)

(21) Appl. No.: 15/706,633

(22) Filed: Sep. 15, 2017

(51) Int. Cl. A01K 11/00; A61B 5/00; G06Q 50/02; A01K 29/00

(52) U.S. Cl. CPC A01K 11/004; A61B 5/0008; A01K 29/00; G06Q 50/02; A01K 11/006; A61B 5/0015

(57) ABSTRACT

Embodiments disclosed herein include an intelligent health monitoring engine comprising: a processor; a memory; a communications interface to receive data from a remote intelligent health monitoring device; a non-transitory computer readable medium storing machine readable instructions that, when executed by the processor, cause the intelligent health monitoring engine to: acquire temperature data associated with an animal based on one or more temperature measures obtained by remote intelligent health monitoring device; determine whether the temperature data acquired satisfies a health condition criteria; identify a treatment modality to be delivered to the animal to treat a health condition if a health condition criteria has been satisfied; identify a caregiver for the animal; generate a data representation for display on a remote computing device running an animal management application having a graphical user interface accessible to the identified caregiver, the data representation including identification information for the animal that needs the treatment modality.

### TECHNICAL FIELD

The disclosed technology relates generally to animal healthcare, and more particularly some embodiments of the present disclosure relate to systems, methods and apparatus for monitoring animal health conditions.

### BACKGROUND

Like humans, animals experience disease, injury, illness, and other health conditions. In providing care for animals, people often wish to treat such health conditions to assist the animal in its recovery. Particularly for those whose livelihood depends on the survival of the animals they care for—e.g., farmers, ranchers, breeders, etc. —the health of animals under their care is of utmost concern. In many instances, to avoid certain diseases, caregivers may proactively vaccinate their animals to avoid serious illness and/or death that may occur if the animals are left unvaccinated. Such owners also often treat their herds with other drugs to help them combat various illnesses. This is especially common in the farming and ranching industries, where the longevity of animal's life directly correlate to the financial success of the farming or ranching operation. Because farmers and ranchers don't know which one or more animals in a given herd will come down with an illness, for example, they often treat their entire herd with the medications they would need to help them avoid the same. Such medications can be very expensive, directly affecting a farmer's profit margins.

The present disclosure, in accordance with one or more various embodiments, is directed toward enhanced technical solutions that inform owners (or others animal caregivers) about the health condition of their animals, on a real-time or near real-time basis, allowing the farmers to avoid having to treat an entire herd with a given medication, and instead only treat those animals that show signs of illness. The solutions disclosed herein, in accordance with one or more various embodiments, reduce animal fatalities, streamline diagnosis and treatment of animal illness, reduce the amount of medication or other drugs that animals (e.g., livestock) are exposed to throughout their lifetime, and save farmers and ranchers money throughout the lifespan of the animals in their herd. As detailed herein, embodiments of the present disclosure may include systems, methods and apparatus for intelligently monitor the health condition of animals, intelligently identify likely health conditions developing within a given animal based on various factors, and intelligently identify a treatment plan and solution for resolving the health condition.

### BRIEF SUMMARY OF EMBODIMENTS

According to various embodiments of the disclosed technology, an intelligent health monitoring engine may include one or more of: a processor; a memory; a communications interface to receive data from a remote intelligent health monitoring device; a non-transitory computer readable medium storing machine readable instructions that, when executed by the processor, cause the intelligent health monitoring engine to: acquire temperature data associated with an animal based on one or more temperature measures obtained by remote intelligent health monitoring device; determine whether the temperature data acquired satisfies a health condition criteria; identify a treatment modality to be delivered to the animal to treat a health condition if a health condition criteria has been satisfied; identify a caregiver for the animal; and provide treatment modality information to the caregiver.

In some embodiments the non-transitory computer readable medium further stores machine readable instructions that, when executed by the processor, cause the intelligent health monitoring engine to: the generate a data representation for display on a remote computing device running an animal management application having a graphical user interface accessible to the identified caregiver, the data representation including identification information for the animal to which the treatment modality is to be delivered; and/or providing the data representation for display on the remote computing device running the animal management application. In some embodiments the data representation includes one or more of a pull list, a non-conforming list, a consecutive missed list.

In some embodiments, the remote intelligent health monitoring device is attached to the ear of the animal, and the temperature data is based on heat sensed within the ear canal of the animal. In some embodiments, the health condition criteria comprises a temperature threshold. In some embodiments, determining whether temperature data satisfies a health condition criteria is based upon one or more of the ambient temperature near the animal's location, the length of time during which the temperature data satisfied the health condition criteria.

In some embodiments, identifying a treatment modality to be delivered to the animal to treat a health condition is based on one or more of: the animal's breed, age, size, treatment history, and known allergies. In some embodiments, identifying a treatment modality to be delivered to the animal to treat a health condition is based on one or more of: the treatment's availability, effectiveness, and cost.

In some embodiments, identifying a treatment modality to be delivered to the animal to treat a health condition comprises selecting among a plurality of medications to deliver to the animal.

In some embodiments, providing treatment modality information to the caregiver comprises: generating one or more of a text message, an email, or a phone call describing the treatment modality information. In some embodiments, treatment modality information comprises one or more of: a medication type, availability, dosage, dosage schedule, and delivery site.

According to various embodiments of the disclosed technology, an intelligent health monitoring engine may include one or more of: a processor; a memory; a communications interface to receive data from a remote intelligent health monitoring device; a non-transitory computer readable medium storing machine readable instructions that, when executed by the processor, cause the intelligent health monitoring engine to: acquire temperature data associated with an animal based on one or more temperature measures obtained by remote intelligent health monitoring device; determine whether temperature data satisfies a health condition criteria; identify a treatment modality to be delivered to the animal to treat a health condition if a health condition criteria has been satisfied; identify a caregiver for the animal; provide treatment modality information to the caregiver.

In some embodiments, the remote intelligent health monitoring device is attached to the ear of the animal, and the temperature data is based on heat sensed within the ear canal of the animal.

In some embodiments, the health condition criteria comprises a temperature threshold.

In some embodiments, determining whether temperature data satisfies a health condition criteria is based upon one or more of the ambient temperature near the animal's location, the length of time during which the temperature data satisfied the health condition criteria.

In some embodiments, identifying a treatment modality to be delivered to the animal to treat a health condition is based on one or more of: the animal's breed, age, size, treatment history, and known allergies. In some embodiments, identifying a treatment modality to be delivered to the animal to treat a health condition is based on one or more of: the treatment's availability, effectiveness, and cost.

In some embodiments, identifying a treatment modality to be delivered to the animal to treat a health condition comprises selecting among a plurality of medications to deliver to the animal.

In some embodiments, providing treatment modality information to the caregiver comprises: generating one or more of a text message, an email, or a phone call describing the treatment modality information. In some embodiments, treatment modality information comprises one or more of: a medication type, availability, dosage, dosage schedule, and delivery site.

According to various embodiments of the disclosed technology, an intelligent health monitoring engine may include one or more of: a processor; a memory; a communications interface to receive data from a remote intelligent health monitoring device; a non-transitory computer readable medium storing machine readable instructions that, when executed by the processor, cause the intelligent health monitoring engine to: acquire physical parameter data associated with an animal based on one or more physical parameter measures obtained by remote intelligent health monitoring device; determine whether the physical parameter data satisfies a health condition criteria; identify a treatment modality to be delivered to the animal to treat a health condition if a health condition criteria has been satisfied; identify a caregiver for the animal; provide treatment modality information to the ca regiver.

According to various embodiments of the disclosed technology, an intelligent health monitoring device may include one or more of: a housing including a casing member releasably couplable with a base member, wherein the casing member and the base member create an internal cavity when in a coupled configuration; an environment resistant seal disposed between the casing member and the base member, wherein the environment resistant seal is held between the casing member and the base member when the casing member and the base member are in the coupled configuration; one or more studs coupled to a side of the base member, the studs configured to pierce biological tissue; a circuit configured to support electrical connections, the circuit supporting electrical connections between at least a processing engine, a memory, a transmitter, and a power source; wherein the processing engine, memory, transmitter circuit, and power source are operatively coupled together and held within the internal cavity; a conductor operatively coupled to the circuit and a heat sensor, a portion of the conductor disposed within a flexible cord passing through an opening in the housing and extending outside the internal cavity to at least a portion of the heat sensor.

In some embodiments, intelligent health monitoring devices of the present disclosure may include a heat sensor in the form of a thermistor. In some embodiments, the heat sensor is a thermistor having a resolution of less than 3 degrees Fahrenheit.

In some embodiments of the intelligent health monitoring device of the present disclosure, the flexible cord and the heat sensor may be configured to be at least partially disposed within an anatomical orifice of an animal. For example, in some embodiments of the intelligent health monitoring device of the present disclosure, flexible cord and the heat sensor are configured to be at least partially disposed within an ear canal of an animal.

In some embodiments of the intelligent health monitoring device of the present disclosure, the transmitter may be a component member of communications interface circuit. The communications interface circuit may include one or more of a Zigbee compliant communications module, a Bluetooth compliant communications module, a Wi-Fi compliant communications module, and a cellular communications module. In some embodiments of the intelligent health monitoring device, the communications interface circuit can transmit an electromagnetic signal to a remote receiver located over 1000 feet from the intelligent health monitoring device. In some embodiments of the intelligent health monitoring device, the communications interface circuit can transmit an electromagnetic signal through the air to a remote receiver located up to 1500 feet away from the intelligent health monitoring device in any direction.

In some embodiments of the intelligent health monitoring device of the present disclosure, the one or more studs are formed from a rigid material. In some embodiments, the one or more studs are formed from a rigid material that is biocompatible. In some embodiments, the one or more studs include a shaft and a head, the head being pointed so as to be adapted for piercing the biological tissue of an animal.

In some embodiments of the intelligent health monitoring device of the present disclosure, the environment resistant seal is made of a compressible, non-rigid material. In some embodiments, at least a portion of the environment resistant seal comprises one of a thermoplastic elastomer and a thermoset rubber. In some embodiments, at least a portion of the environment resistant seal comprises santoprene.

In some embodiments of the intelligent health monitoring device of the present disclosure, the largest dimension of the base member is less than 3 inches. In some embodiments, the largest dimension of the base member is about between 1.5 and 1.7 inches.

In some embodiments of the intelligent health monitoring device of the present disclosure, the weight of the HMD is less than 33 grams. In some embodiments, the weight of the HMD is between 20 and 30 grams. In some embodiments the weight of the HMD may be greater than or less than 20 grams.

Some embodiments of the intelligent health monitoring device of the present disclosure further comprise one or more of a light source configured to emit light responsive to a signal received from a remote source, and an audio source configured to emit sound responsive to a signal received from a remote source.

In some embodiments of the intelligent health monitoring device of the present disclosure, the power source is one of a rechargeable power source, a removable power source, and a solar power source.

According to various embodiments of the disclosed technology, an intelligent health monitoring device may include one or more of: a casing member releasably couplable with a base member, wherein the casing member and the base member create an enclosure having a single opening when in a coupled configuration; an environment resistant seal disposed between the casing member and the base member, wherein the environment resistant seal is held between the casing member and the base member when the casing member and the base member are in the coupled configuration; one or more studs coupled to a side of the base member and extending at least 5 millimeters from the side of the base member, the studs configured to pierce biological tissue; a circuit held within the enclosure, the circuit comprising: a processing engine, a memory, a transmitter, and a power source, wherein the processing engine, memory, transmitter, and power source are operatively coupled together and held within the enclosure; a conductor operatively coupled to the circuit and a heat sensor, the conductor passing through the opening of the enclosure and leading to at least a portion of the heat sensor held outside the enclosure; and a sleeve coupled to the casing member and surrounding the conductor between the casing member and the portion of the heat sensor held outside the enclosure.

According to various embodiments of the disclosed technology, an intelligent health monitoring device may include one or more of: a casing member releasably couplable with a base member, wherein the casing member and the base member create an enclosure having one or more openings when in a coupled configuration; a moisture resistant seal held between the casing member and the base member when the casing member and the base member are in the coupled configuration; one or more studs coupled to a side of the base member, the studs configured to pierce biological tissue; a circuit held within the enclosure, the circuit including one or more of a processing engine, a memory, a transceiver, and a battery; wherein the processing engine, memory, transceiver, and battery are operatively coupled together and releasably held within the enclosure; and a cord coupled with the circuit one end, the cord including an insulation sleeve surrounding one or more conducting wires, the conducting wires connected to a heat sensor held outside the enclosure.

Some embodiments of the present disclosure include a method for monitoring the health of an animal by obtaining real-time temperature measurements acquired by an intelligent health monitoring device. In accordance with some embodiments, the method includes one or more of the following steps: attaching an intelligent health monitoring device to the ear (or other body part) of an animal; causing the intelligent health monitoring device to obtain a temperature measurement data associated with the animal (e.g., the temperature within the animal's ear canal); transmitting the temperature measurement data from the intelligent health monitoring device to a receiver; receiving at the receiver the temperature measurement data transmitted from the intelligent health monitoring device. In some such embodiments, the intelligent health monitoring device comprises: a casing member releasably couplable with a base member, wherein the casing member and the base member create an enclosure having one or more openings when in a coupled configuration; a moisture resistant seal held between the casing member and the base member when the casing member and the base member are in the coupled configuration; one or more studs coupled to a side of the base member, the studs configured to pierce biological tissue; a circuit held within the enclosure, the circuit comprising a processing engine, a memory, a transceiver, and a battery; wherein the processing engine, memory, transceiver, and battery are operatively coupled together and releasably held within the enclosure; and a cord coupled with the circuit one end, the cord comprising a sleeve surrounding one or more conducting wires, the conducting wires connected to a heat sensor held outside the enclosure.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

Some of the figures included herein illustrate various embodiments of the disclosed technology from different viewing angles. Although the accompanying descriptive text may refer to such views as “top,” “front,” “back,” “bottom” or “side” views, such references are merely descriptive and do not imply or require that the disclosed technology be implemented or used in a particular spatial orientation unless explicitly stated otherwise.

FIG. 1 illustrates a block diagram representing one or more elements of an example system for monitoring animal health condition in accordance with one or more embodiments of the present disclosure.

FIG. 2A illustrates a side view of an example intelligent animal health monitoring device (hereinafter, referred to as an “HMD”) in accordance with one or more embodiments of the present disclosure.

FIG. 2B illustrates a rear side of an example HMD in accordance with one or more embodiments of the present disclosure.

FIG. 2C illustrates a top view of an example HMD in accordance with one or more embodiments of the present disclosure.

FIG. 2D illustrates a bottom view of an example HMD in accordance with one or more embodiments of the present disclosure.

FIG. 3 illustrates an exploded perspective view of an example HMD in accordance with one or more embodiments of the present disclosure.

FIG. 4 illustrates an exploded perspective view of another example HMD in accordance with one or more embodiments of the present disclosure.

FIG. 5 illustrates a block diagram representing various electronic components of an example HMD in accordance with one or more embodiments of the present disclosure.

FIG. 6 illustrates an example HMD attached to an animal, in this depiction attached to an ear of a cow in accordance with one or more embodiments of the present disclosure.

FIG. 7A illustrates an environment within which one or more embodiments of the systems, methods, and apparatus of the present disclosure may be implemented.

FIG. 7B illustrates the environment depicted in 7A, here demarking example zones for effective signal transmission to or from one or more of the receivers that may be deployed in one or more embodiments of the present disclosure.

FIG. 7C illustrates the environment depicted in 7B, here depicting one or more animals within the monitored area, and demarking the zones within which some of those animals are located, in accordance with one or more embodiments of the present disclosure.

FIG. 8 illustrates an example computing platform that may be implemented in accordance with one or more embodiments of the present disclosure.

FIG. 9 illustrates various other components that may be implemented in accordance with one or more embodiments of the present disclosure.

FIG. 10 illustrates an example login input field in a GUI (e.g., of a web portal or an AMM App) that may be implemented at a client computing device in accordance with one or more embodiments of the present disclosure.

FIG. 11 illustrates an example dashboard and various data representations (e.g. a pull list), buttons, tabs, and options available via a GUI (e.g., of a web portal or an AMM App) that may be implemented in accordance with one or more embodiments of the present disclosure.

FIG. 12 illustrates an example chart and various data representations, buttons, tabs, and options available via a GUI (e.g., of a web portal or an AMM App) that may be implemented in accordance with one or more embodiments of the present disclosure.

FIG. 13 illustrates an example chart and various data representations, buttons, tabs, and options available via a GUI (e.g., of a web portal or an AMM App) that may be implemented in accordance with one or more embodiments of the present disclosure.

FIG. 14 illustrates an example chart and various buttons, tabs, and options available via a GUI (e.g., of a web portal or an AMM App) that may be implemented in accordance with one or more embodiments of the present disclosure.

FIG. 15 illustrates various example data representations, buttons, tabs, and options available via a GUI (e.g., of a web portal or an AMM App) that may be implemented in accordance with one or more embodiments of the present disclosure.

FIG. 16 illustrates various example data representations, buttons, tabs, and options available via a GUI (e.g., of a web portal or an AMM App) that may be implemented in accordance with one or more embodiments of the present disclosure.

FIG. 17 illustrates various example data representations, buttons, tabs, and options available via a GUI (e.g., of a web portal or an AMM App) that may be implemented in accordance with one or more embodiments of the present disclosure.

FIG. 18 illustrates various example data representations, buttons, tabs, and options available via a GUI (e.g., of a web portal or an AMM App) that may be implemented in accordance with one or more embodiments of the present disclosure.

FIG. 19 illustrates various example data representations, buttons, tabs, and options available via a GUI (e.g., of a web portal or an AMM App) that may be implemented in accordance with one or more embodiments of the present disclosure.

FIGS. 20A-20C illustrate various example data representations, buttons, tabs, and options available via a GUI (e.g., of a web portal or an AMM App) that may be implemented in accordance with one or more embodiments of the present disclosure.

FIG. 21 illustrates various example data representations, buttons, tabs, and options available via a GUI (e.g., of a web portal or an AMM App) that may be implemented in accordance with one or more embodiments of the present disclosure.

FIGS. 22A-22B illustrate an example client computing device running an example AMM App, here depicting example Pull List (e.g., Treat/Retreat list), in accordance with one or more embodiments of the present disclosure.

FIG. 23 illustrates an example client computing device running an example AMM App, here depicting an example Non-Conforming List, in accordance with one or more embodiments of the present disclosure.

FIG. 24 illustrates a symbolic diagram depicting an example hierarchical organization of caregivers within an example operation, including an example of various user categories, in accordance with one or more embodiments of the present disclosure.

The figures are not intended to be exhaustive or to limit the disclosed technology to the precise form disclosed. It should be understood that the invention can be practiced with modification and alteration, and that the disclosed technology be limited only by the claims and the equivalents thereof.

### DETAILED DESCRIPTION OF THE EMBODIMENTS

Embodiments of the present disclosure enable animal caregivers to more precisely, intelligently, and timely monitor the health of the animals they care for, and to more effectively and efficiently treat animals that exhibit symptoms or other indications of a developing (or already developed) health condition. The technology disclosed herein provides animal caregivers with the tools they need to collect, access, interpret, and interact with information (real-time information, historical information, etc.) about animals under their care, and further to effectively manage tasks, assignments, and procedures that help facilitate treatment, transfer, or termination of animals under their care.

The systems, methods, and apparatus for realizing the various benefits of the presently disclosed technology may be tailored, and in some embodiments customizable, to the individual needs of a given animal caregiver's operation. Such tailoring and customization may be made on the basis of one or more factors of import/relevance to a particular animal caregiver (e.g., a handler's skillset), a particular animal or group of animals (e.g., particular breeds, groups, etc.), or the operation generally (e.g., cost, efficiency, staffing etc.). For example, in environments where a caregiver's operation includes multiple parties (e.g., managers, supervisors, animal handlers, etc.) responsible for one or more of the animals under care of the operation, the presently disclosed technology may be implemented to provide one or more such party (e.g., each of the employees on a farming operation) with access to one or more of the features disclosed herein (e.g., an animal handler might access information relevant to animals under his care by logging into a web portal or viewing information relevant to him/her on a mobile app running on their smartphone). In some embodiments, a given user's ability to utilize one or more features may be limited by their role in the operation (e.g., a supervisor may be given greater access or task delegating authority than a handler, for example).

Several example implementations of the present technology are discussed within this disclosure for the benefit of the reader's understanding, it being understood that these example implementations are in no way intended to limit the robust features that the present disclosure teaches or suggests. The example implementations disclosed herein are instead intended to illustrate various utilities of the present technology, to provide examples of how the presently disclosed technology may be tailored or customized for operation in a particular environment, to assist the reader in understanding the various contexts within which the present technology may be deployed, and ultimately to provide added clarity and enhanced understanding of the technology disclosed herein.

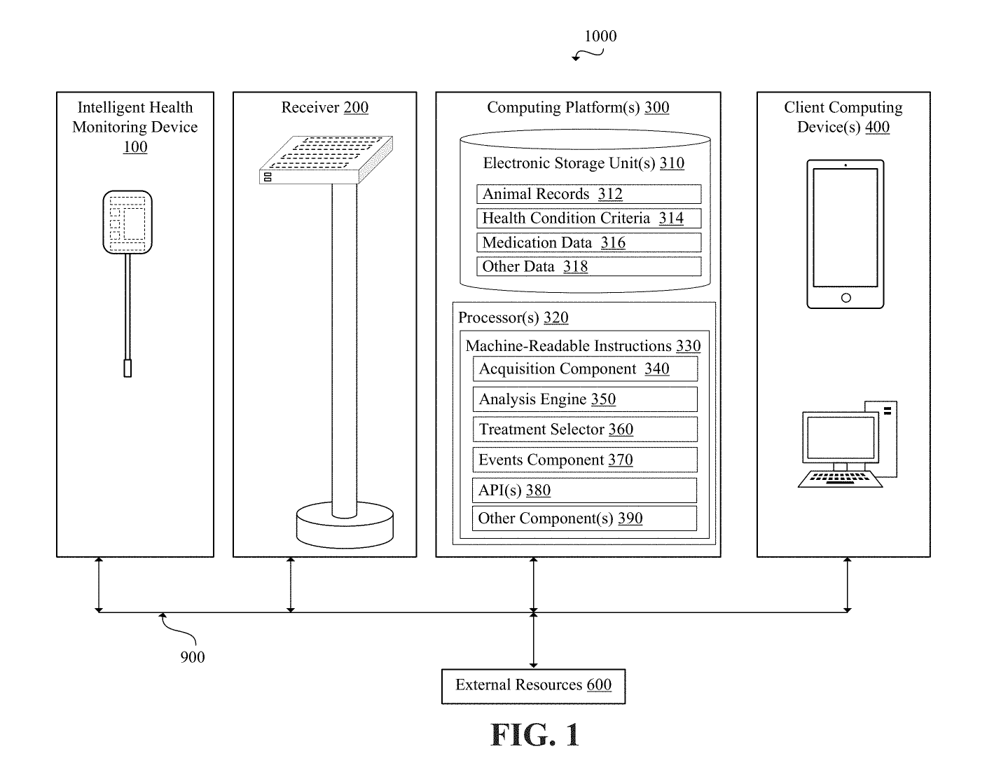

FIG. 1 illustrates a block diagram representing one or more elements of an example system for monitoring animal health conditions in accordance with one or more embodiments of the present disclosure. As shown, system 1000 may include one or more intelligent animal health monitoring devices 100 (hereafter, HMDs 100), one or more receivers 200, one or more computing platforms 300, and one or more client computing devices 400.

In some embodiments, HMD 100 is attachable to or otherwise couplable with the body of an animal. HMD may include hardware and software (described in more detail below) that senses one or more physical parameters associated with the animal (e.g., temperature inside the animal's ear canal) or the animal's environment (e.g., ambient temperature at the animal's location) and effectuates transmission of one or more signals representing (or enveloping) information associated with the sensed physical parameters.

In some embodiments, the HMD 100's signal transmission is received directly by the computing platform 300, and in other embodiments the one or more signals are relayed through one or more other nodes before the information arrives at the computing platform 300. In some embodiments, the one or more other nodes perform one or more processing or preprocessing operations on the information before sending it along to the computing platform 300. For example, one or more of the signals transmitted by an HMD 100 may be received by one or more receivers 200 placed within geographical range of the HMD 100's transmission capacity. The receivers 200 may be in communication with one or more concentrators (not shown) which may obtain the information received by receivers 200 and may perform some concentration operations on the information before passing it to the computing platform 300. For example, such concentration operations may include filtering erroneous data, establishing a relationship between multiple readings taken over a given time period for a given animal, reformatting the information for storage in an electronic storage media or for entry into a database (which in some embodiments may be part of computing platform 300), or otherwise. At a high level, it should be understood that any one or more of the nodes (when used in a given implementation) may perform some processing or preprocessing operations on any of the information available to system 1000. Regardless of the arrangement, ultimately information associated with the physical parameters measured/sensed by an HMD 100 may be conveyed to, processed, and/or stored at computing platform 300, whether in the same or different format as provided in an original transmission.

In some embodiments, the one or more receivers 200 decode the signals received from the one or more HMDs 100 to obtain the information (also referred to herein as data) they carry, and provide the information to one or more computing platforms 300 of system 1000. The one or more computing platforms 300 may receive the data (or signals representing the data) from the one or more receivers 200 for storage, and perform analysis or further operations on that data to effectuate one or more of the features disclosed herein.

As explained in more detail below, in some embodiments, computing platform uses the data obtained via receivers 200 to detect the onset of a developing (or already developed) health condition in a given animal, and in some instances to determine an appropriate treatment modality for the given animal based on information available to system 1000. In some embodiments, if the one or more computing platforms 300 determine that a particular treatment or treatments is/are appropriate or otherwise recommended for a given animal based on the information available to system 1000, the one or more computing platforms 300 may provide details about the animal, the health condition, the recommended treatment, or other relevant information to (or make such treatment information accessible by) one or more client computing devices 400 (e.g., remote desktop computers, smartphones, tablets, etc.) associated with one or more caregivers overseeing the care of the given animal.

As symbolically shown in FIG. 1, any one or more of the elements in the system may communicate with (e.g., be operatively coupled with, or be otherwise accessible to) any of the other elements or subelements of system 1000. As shown, such communication may take place over one or more communication links 900. Communications links 900 may take the form of any wired or wireless communications interface technology desired (including associated hardware, software, protocols, etc.), including any such communications interface technologies already known in the art, as well as any later developed. It should further be understood that some embodiments of the present disclosure may not include each element depicted in FIG. 1, and some embodiments may include more elements (e.g., additional transmission nodes beyond receivers 200) or features than those depicted in FIG. 1. Because elements within system 1000 may be equipped with one or more communications interfaces, such elements may, in addition to being in communication with one another, also be in communication with various other external resources over communications links 900. In some embodiments, the various external resources (e.g., external databases accessible over the Internet) may be utilized by system 1000, or as part of system 1000, for additional information, computing power and enhanced functionality of system 1000.

Some example embodiments, which may be implemented via one or more of the elements depicted in FIG. 1, will now be discussed in more detail to provide additional clarity and understanding.

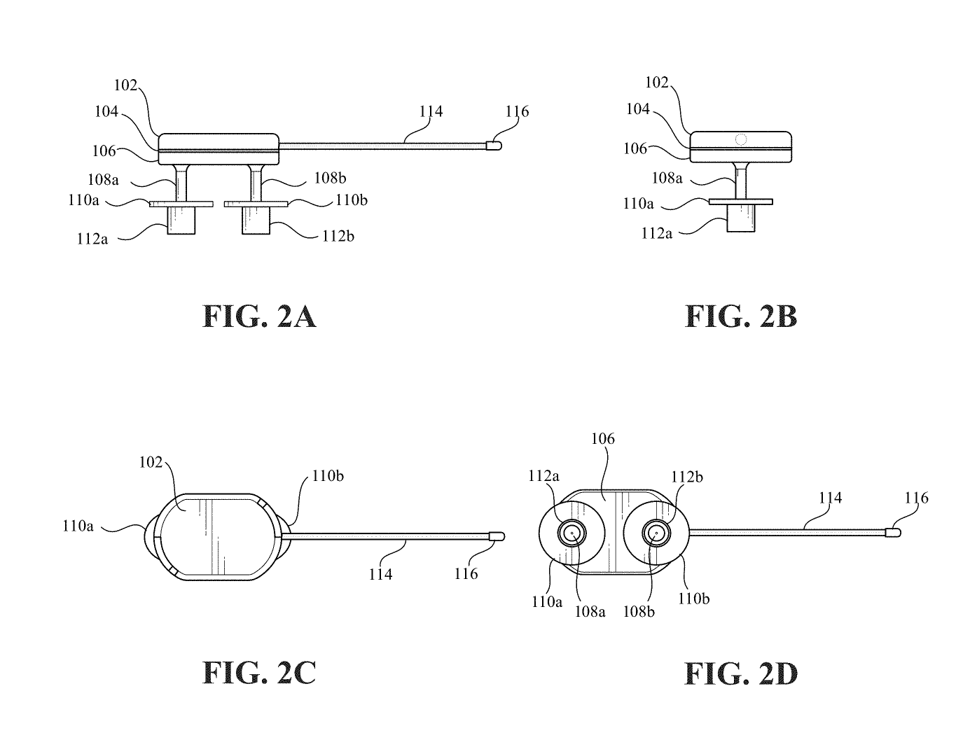

FIGS. 2A-2D illustrate various views of an example HMD 100 in accordance with one or more embodiments of the present disclosure. FIG. 2A illustrates a side view of the exterior of an example HMD 100 in accordance with one or more embodiments of the present disclosure. FIG. 2B illustrates a rear side of the exterior of an example HMD 100 in accordance with one or more embodiments of the present disclosure. FIG. 2C illustrates a top view of the exterior of an example HMD 100 in accordance with one or more embodiments of the present disclosure. FIG. 2D illustrates a bottom view of the exterior of an example HMD 100 in accordance with one or more embodiments of the present disclosure. FIGS. 2A-2D will be discussed together, with like numerals referring to like elements of the example HMD 100 embodiment shown.

As shown in FIGS. 2A-2D, HMD 100 may include a housing including a casing member 102 (sometimes referred to herein as a first member) releasably coupled with a base member 106 (sometimes referred to herein as a second member). When coupled together, casing member 102 and base member 102 may define an internal cavity wherein other components (e.g., electronic hardware and software components) may be disposed.

As shown, HMD 100 may include an environment resistant seal 104. Environment resistant seal 104 may be disposed between at least a portion of casing member 102 and at least a portion of base member 106 to provide a barrier between the interior cavity and the external environment when the casing member 102 and base member 106 are in a coupled position. Environment resistant seal 104 may be provided by any one or more materials, or combinations of materials, to provide the barrier desired. For example, in some instances liquid resistance may be desired; in some instances gaseous flow resistance may be desired; in some instances temperature fluctuation resistance may be desired; in some instances fire resistance may be desired. In such examples, respectively environment resistant seal 104 may be provided by a waterproof material, gas-impermeable material, temperature insulating material; or a fire resistant material. One of ordinary skill in the art will appreciate that various other materials may be used, depending on the application and desired resistive objective, to provide at least partial protection from external environmental elements to interior components held in the interior cavity of the HMD 100 housing. In many applications, it may be desirable to resist many features of an external environment (e.g., to inhibit dust, water, fluid, and other particulates of the external environment) from entering the internal cavity area of the HMD 100 housing. In such embodiments a composite material with multifunctional properties may be used as the environment resistant seal 104. In some embodiments the environment resistant seal 104 may be provided, in whole or in part, by a santoprene material.

In some embodiments, the environment resistant seal 104 provides protection from environmental elements to the interior components held inside the interior of the housing of HMD 100. In some embodiments, environment resistant seal 104 provides a substantially waterproof seal along an edge of a side wall of the casing member 102. In some embodiments, environment resistant seal 104 provides a substantially waterproof seal along an edge of a side wall of the base member 106. In some embodiments, at least a portion of the environment resistant seal 104 is a soft or flexible material (e.g., santoprene) that is subject to compression upon incident compressive forces. In some embodiments a portion of the environment resistant seal 104 may be pinched (and consequently compressed) between the casing member 102 and base member 106 as casing member 102 and base member 106 are brought together and releasably coupled together.

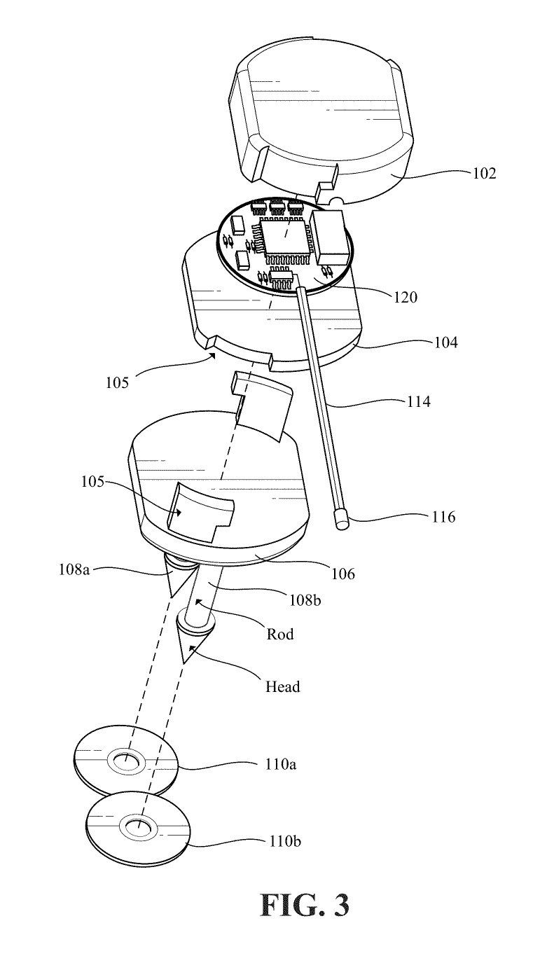

As shown, HMD 100 may include one or more studs 108 (e.g., stud 108a, stud 108b) to provide an attachment mechanism to install the HMD 100 onto the body of an animal. HMD 100 may further include one or more fittings 110 (e.g., fitting 110a, fitting 110b) that provides a releasable couple with the one or more studs 108. In some embodiments, the one or more fittings 110 may include a cap 112 (e.g., cap 112a, cap 112b) to protect or otherwise guard the distal end of the studs (which may in some embodiments be pointed/sharpened as shown in FIG. 3). In some embodiments the fitting 110 does not include a cap 112 portion, and the distal end of the studs 108 is exposed thorough the outward facing side of the fitting 110. In some embodiments, the one or more studs 108 may include a sharpened portion adapted to pierce through the surface of an animal's skin to realize the attachment. Such sharpened portion is not shown in FIGS. 2A-2D (as such is covered by the cap 112 portion of fittings 110), but an example of such is shown in FIG. 3. As shown in FIG. 3, the one or more studs 108 may be sharpened at a distal end (i.e., the end opposite the end that is coupled to base member 106) so that an operator may pierce the ear (or other surface) of an animal to attach the HMD 100 to the body of the animal, for example. In some embodiments the studs 108 are used to attach the HMD 100 to another device (e.g., a belt, a harness, helmet etc.) that is attached to the animal such that the HMD 100 may be installed without piercing the skin of the animal. To avoid deformation of the studs 108 over time (e.g., by bending inward or outward with extended use), high strength materials may be used to form the rod or shaft of the studs 108. For example, some non-limiting embodiments may include studs formed, in whole or in part, of a material including a rigid nylon ABS blend. In still further embodiments, the structural dimensions of the studs may be configured to avoid bending. For instance, in some embodiments, such as those where the studs 108 are intended for piercing the ear of a cow, the diameter (or largest cross-sectional dimension, if not annular) of the studs may be between 2 and 10 millimeters, or greater. In some embodiments, the ratio of the diameter (or largest cross-sectional dimension, if not annular) of the studs to the length of the stud rod or shaft is greater than 0.05:1 but less than 1:1.

In still further embodiments, some HMD 100's may be implemented without studs, but instead utilize a clip or other attachment mechanism (not shown) to couple, releasably or permanently, the HMD 100 (or a portion of the HMD) to the body an animal itself or another device (e.g., a belt, a harness, helmet, etc.) that is attached or attachable to the animal.

As shown in FIGS. 2A-2D, HMD 100 may include a thermistor 116 (or other sensor or sensor array, as desired for the given application) disposed at or near the end of a flexible cord 114 that extends outside the interior cavity of the HMD 100. In some embodiments, thermistor 116 is connected to electronic componentry held in the interior cavity of HMD 100 such that the temperature readings detected by thermistor 116 may be communicated to (e.g., via an electric signal generated as the sensor transduces the physical parameter detected), processed by, stored upon, and/or transmitted by HMD 100. In some embodiments, as shown, the connection between thermistor 116 and the electronic componentry held in the interior cavity of the HMD 100 housing is a wired connection (e.g., provided by one or more wires held within cord 114, the wire(s) traversing through a side wall of either or both the casing member 102 and the base member 106 and tying into the aforementioned electronic componentry (e.g., soldered to a connection point on a circuit board held inside the interior cavity of HMD 100.

In some embodiments, the length that the flexible cord 114 extends outside the housing of the HMD is adjustable. For instance, the channel through a side wall of the HMD 100 housing may be sized such that the outer surface of cord 114 is substantially gripped by the inside walls of the channel, substantially securing the cord 114 in place unless and until a user opens the housing (e.g., releasing the releasable couple between the casing member 102 and the base member 106) and applies a force to the cord that draws additional length of the cord 114 into the housing (such that the portion extending outside the housing is shortened) or draws additional length of the cord 114 outside of the housing (such that the portion extending outside the housing is lengthened). Thus, according to some embodiments, the length of the cord 114 may be adjusted to account for differences in the size of the animal with which the HMD 100 is being used, or the size of the space within which the cord 114 is used.

Referring still to FIGS. 2A-2D, although HMD 100 is depicted in this example embodiment to be equipped with a thermistor 116, one of skill in the art should appreciate that HMD 100 may be equipped with any one or more sensors, or an array of sensors, adapted to detect any one or more physical parameters about the animal to which the HMD 100 is associated, or about the environment within which said animal is located. Indeed, any section of the present disclosure that makes reference to a thermistor 116 should also be understood to be equally applicable to any other sensory device, effectively replacing [thermistor] 116 with any other [sensor name] 116 desired for the given application. It should be further understood that such sensors may be disposed inside the interior cavity of the HMD 100, outside of the interior cavity of the HMD 100, and in some instances may be partially disposed inside and partially disposed outside the interior cavity of the HMD 100. In some such embodiments the portion of the sensor device disposed partially outside the interior cavity of the HMD 100 may be connected, via a wired or wireless connection, with the complementary portion of the sensor device disposed within the interior cavity of the HMD 100.

With reference to the releasable couple feature of the HMD 100, the releasable couple may be provided using any mechanism, including any known in the art and any later developed. Such releasable couple provides operators with quick and easy access the internal electronic components (discussed in more detail below). Releasable couples may be used to enable operators to quickly and easily take apart or otherwise detach individual pieces of the HMD 100 from one another for repair, replacement, cleaning or otherwise. Indeed, in some embodiments it may be desirable to separate one part of the HMD 100 from another part of the HMD 100 for a time, and also to have the ability to recouple said parts back together at a later time. Some examples of such releasable couple mechanisms may include snap-fit (e.g., torsional, cantilever, annular, etc.), twist-fit (e.g., threaded), pressurized (e.g., adjustable valve regulated hermetic seal), friction-fit, and the like.

For example, if an HMD 100 is discovered to be malfunctioning, an operator may wish to remove the casing member 102 (containing or otherwise concealing the damaged electronic components, for example) from the base member 106 (which may be pierced through the animal's ear, for example) so that the operator can obtain with very little effort (by simply uncoupling the casing member 102 from the base member 106, for example) the internal pieces he/she needs to repair, replace, clean or otherwise.

Releasable couple mechanisms may be deployed between any one or more hardware elements of an HMD 100 (in addition to the casing member 102 and the base member 106 discussed above) to enable quick and easy separation of one part of the HMD 100 from another as desired. Taking the example in the previous paragraph even further, the HMD 100 may include electronic componentry tied into a circuit (e.g. a printed circuit board (“PCB”)), adapted to be held at least partially within the a cavity region defined by the walls of the casing member 102 of the HMD 100 housing. For ease of description throughout this disclosure, any electronic circuit boards, including printed circuit boards, integrated circuits, or other circuit arrangements/formats, will generally be referred to herein as PCBs, it being understood that in some embodiments the circuitry deployed therein needn't actually take the form of a printed circuit board. With that understanding, and by way of a nonlimiting example, the PCB itself may be releasably coupled to an interior wall of the casing member 102 so that the PCB may be securely positioned when coupled. Thus, in some implementations an operator may uncouple the casing member 102 from the base member 106 to get at the PCB held inside, then further uncouple the PCB from the casing member 102 by disengaging a releasable couple mechanism therebetween such that the PCB may be separated from an interior wall of the casing member 102 and taken to a remote location for repair. In some such instances, once the PCB is removed, the operator may wish to recouple the casing member 102 of the HMD 100 with the base member 102 so as to keep the remaining parts together until the repaired PCB is ready to be reinstalled (i.e., recoupled with the casing member 102) for further operation within system 1000.

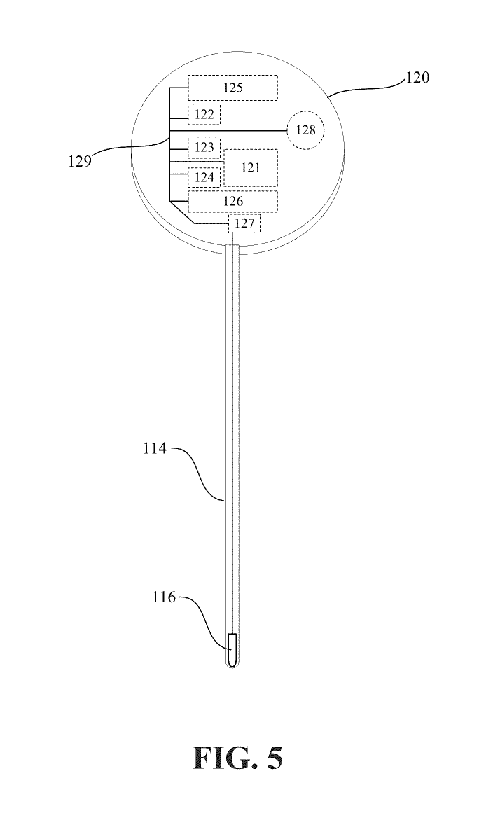

As shown in FIGS. 2A-2D, HMD 100 may include a cord 114 connecting thermistor 114 to the housing of HMD 100 (and the electronic componentry held therein). In some embodiments, the cord is made of a non-rigid (i.e., substantially flexible) material such as a medical grade polymer material. The cord 114 may in some embodiments be a sleeve (e.g., a tubular structure) that loosely covers wiring between the thermistor and the housing of the HMD 100 (instead of being molded or formed around the conducting material of the wire itself). The cord 114 may in some embodiments be a sleeve that covers wiring between the thermistor and the housing of the HMD 100, and in some embodiments the sleeve may also include a closed distal end (like a tube closed on one end), the thermistor (or other sensor) being enclosed entirely within the sleeve such that the thermistor 116 not exposed to the external elements of the surrounding environment, but is situated within the sleeve such that it may still sense external parameters (such embodiment is shown in FIG. 5). The cord 114 may be provided with a length and width appropriate to guide (and in some instanced hold or secure) the thermistor 116 at or near the proper location on the animal to obtain the desired reading.

For example, if the temperature within the ear canal of an adult cow of a particular size is the desired measure sought by the animal caregiver, cord 114 may be about between 2-4 inches in length, and less than ¼ inch in diameter (or other width dimension depending on the cross-sectional profile of the cord) such that HMD 100 may installed onto the adult cow's ear (e.g., with studs 108a and 108b piercing through the cow's ear at a location on the cow's ear such that the cord 114 can be directed into and remain disposed within the ear canal of the cow (until forcibly removed) to obtain the desired temperature reading(s) the operator is interested in. The foregoing is a specific and non-limiting example, and one of skill in the art will appreciate that the embodiments described herein may be modified in various manners (e.g., different sensors, different HMD attachment locations, different physical parameters measured, etc.) without departing from the scope and spirit of the present disclosure.

FIG. 3 illustrates an exploded perspective view of another example HMD 100 in accordance with one or more embodiments of the present disclosure. As shown, HMD 100 may include a casing member 102 defining at least a portion of a cavity within which an electronic circuit board 120 may be at least partially disposed. HMD 100 may also include an environment resistant seal 104 to provide at least partial protection from environmental elements to interior components held inside the interior cavity of the housing of HMD 100—e.g., to inhibit dust, water, and other particulates of the external environment from entering the internal cavity area of the HMD 100 housing.

In the example embodiment shown in FIG. 3, environment resistant seal 104 is notched (e.g., see notched region 105, for example) to accommodate the releasable coupling features (e.g., see releasable coupling feature 107, for example) that enable casing member 102 and base member 106 to be releasably coupled together. Also in the example embodiment shown, environment resistant seal 104 is configured to cover or otherwise overlay the cavity created by the structure of casing member 102, thereby spanning from sidewall edge to sidewall edge of casing member 102 and creating a layer or barrier between casing member 102 and base member 106. As with all the features described in the present disclosure, though neither of the foregoing features need be present in embodiments of the presently disclosed technology, in some instances they may be present.

As shown, base member 106 includes or is otherwise coupled with two studs 108, namely stud 108a and stud 108b. Studs 108 may be formed as rods coupled to and extending outward from base member 106. Studs 108 may have sharp or pointed heads (e.g., conical shaped heads as shown in FIG. 3) that create a lip or shelf designed to prevent a pierced surface from slipping off the stud without considerable force (e.g., intentional force). As shown, the lip is formed by the base of the conical heads of the studs 108, the base of the conical heads extending radially outward from the rod structure of studs 108, the lip arising on account of the conical head's base having a diameter larger than the diameter (or other cross sectional width dimension depending on the cross-sectional profile of the stud 108) of at least a portion of the rod structure of studs 108. In some embodiments, the lip or shelf can reduce the chance of, or even prevent, a pierced surface from slipping off of the stud without considerable force. This feature helps keep the HMD 100 attached to the animal until it is intentionally removed.

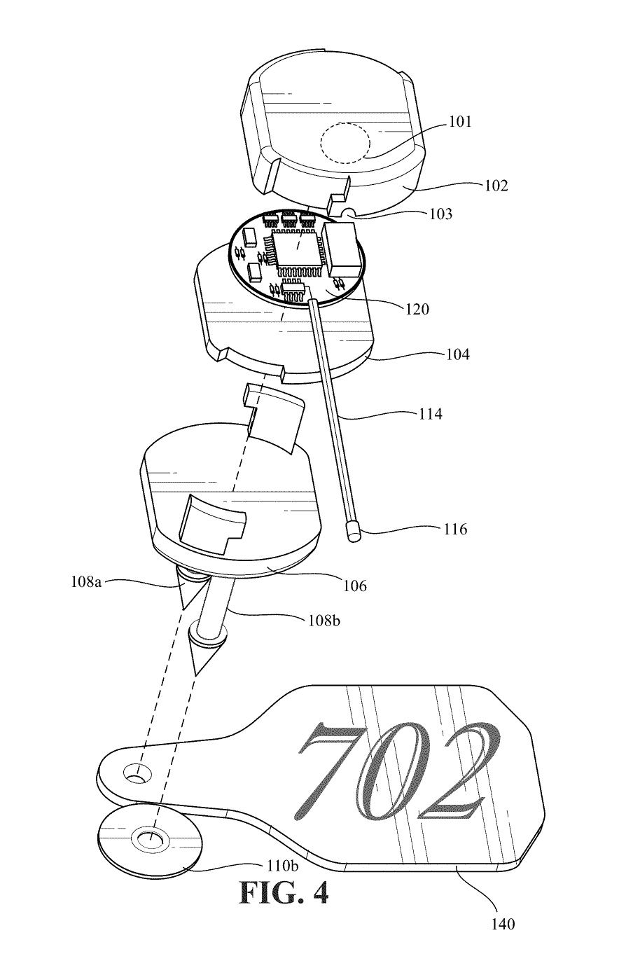

Though depicted with a lip or shelf structure in FIG. 3, some embodiments may be implemented without such a feature. Indeed, in some embodiments, studs 108 may simply comprise a rod or shaft with a flat distal end, or even a sharpened distal end that doesn't create a shelf with the rod structure. In some embodiments, studs 108 may include a feature designed for complementary releasable coupling with a fitting. Such feature may include a shelf, or any other feature (e.g., a ball joint that slips into a releasable complementary case). In embodiments, studs 108 may be implemented with a combination of any one or more of the aforementioned features. For example, as shown in FIG. 3, HMD 100 may include studs 108 that are both sharp at a distal end, shelfed to avoid slip-off, and fitted for releasable coupling with one or more complementary fittings 110, namely fitting 110a and fitting 110b, as shown in FIG. 3. Fittings 110 may serve to further reduce the chance of, or further ensure the prevention of, a pierced surface from slipping off the stud 108 without considerable force. Such fittings may be particularly useful when operators desire to secure other items to HMD 100 via one or more of studs 108, such as an ear tag identifier 140 as shown in FIG. 4. In alternative embodiments, the tag identifier 140 itself may be configured or otherwise formed with a structure (e.g., an complementary sized opening and suitable material) similar to a fitting 110 such that an ear tag identifier 140 may serve as both a fitting and an identifier (avoiding the need in FIG. 4 for the fitting 110a shown in FIG. 3, in some embodiments.

FIG. 4 illustrates an exploded perspective view of another example HMD 100 in accordance with one or more embodiments of the present disclosure, here depicted with an ear tag identifier 140 having a hole through which one or more of studs 108 may pass. This way, studs 108 of HMD 100 can be used as a mechanism to attach other items (e.g., an ear tag identifier 140) that may aid an operator in maintaining an organized operation.

In addition to the ear tag identifier 140, the example embodiment shown in FIG. 4 is different from the embodiment shown in FIG. 3 in other ways. As shown in FIG. 4, HMD 100 may include a light source 101 (e.g. an LED, a laser beam generator, or other light source). The light source may be configured to emit light, which may in some embodiments pass through one or more openings, apertures, or windows (e.g., covered by glass, plastic, or otherwise) to provide one or more visual indications to an operator or other animal caregiver.

For instance, suppose an operator has a herd of 5,000 cattle roaming in a field, and system 1000 has notified him that cow number 702 has developed a particular health condition and needs to be pulled from the herd so that it may be treated a particular antibiotic. System 1000 may automatically, or upon request by an end user (such as the operator, handler, or other caregiver etc.) cause the light source 101 of cow number 702's HMD 100 to flash a red light, for example. This way, especially in the evening hours or in areas with low lighting, the operator may more easily locate cow number 702 among the herd as they are roaming in the field. The end user merely needs to look for the flashing red light among the herd. In some embodiments the light parameters (e.g., frequency, wavelength, intensity, and emission pattern or color) may be adjustable or set by default. Settings for the light source may be stored in a memory of the HMD 100.

In still further embodiments, HMD 100 may include an audio source (e.g. a speaker). The audio source may be configured to emit sound, which may in some instances be heard outside the exterior of HMD 100's housing to provide one or more audible indications to the operator. For instance, extending the example above where an operator has a herd of 975 cattle roaming in a field, and system 1000 has notified him that cow number 702 has developed a particular health condition and needs to be pulled from the herd so that it may be treated with a particular antibiotic. System 1000 may automatically, or upon request by an end user (such as the operator, handler, etc.) cause the audio source of cow number 702's HMD 100 to beep periodically, for example. This way, especially during evening hours or in areas with low lighting, the operator may more easily locate cow number 702 among the herd as they are roaming in the field. The end user merely needs to listen for the beeping sound coming from cow number 702's HMD 100 and then follow the sound to the source. The beeping sound may also be adapted to emit a frequency the creates an annoyance for the cattle, thereby causing other cattle that may be surrounding cow number 702 to walk away—dividing cow number 702 from the herd for easy capture. Once the herd of cattle are separated from cow number 702, for example, if the beeping sound is causing cow number 702 to bustle (in an effort to escape the irritating sound) such that the cow is difficult to capture, the end user may selectively turn the sound off (e.g., using a handheld client computing device 400, remote, etc.) to help the animal settle down. In some embodiments the audio parameters (e.g., frequency, volume, pitch, and emission pattern) may be adjustable or set by default. Settings for the audio source may be stored in a memory of the HMD 100.

FIG. 5 illustrates a block diagram representing various electronic components that may be implemented in an example HMD in accordance with one or more embodiments of the present disclosure. As shown, one or more electronic circuit boards 120 of an HMD 100 may include one or more of a power source 121, a processing engine 122, a memory 123, a communications interface 124, an audio source 126, a light source 128, and sensor circuitry 127 operatively coupled with thermistor 114 over a wire within cable 116 (here cable 116 takes the form of a sleeve that loosely surrounds the wire and encloses the thermistor 116 within the hollow of the sleeve), and one or more other components 125. Though depicted with sensor circuitry 127 operatively coupled to a thermistor 114 over a wire within cable 116, HMDs 100 of the present disclosure may include any one or more sensors as an alternative or in addition to a thermistor (whether disposed entirely or partially inside of or exterior to the internal cavity of HMD 100's housing) for measuring physical parameters associated with the animal or the environment within which the animal is located. Such sensors may include, but are not limited to, one or more of light sensors (e.g., photodetectors for use with, by way of example, oximeters, retinal scanners, etc.), altitude sensors, pressure sensors, moisture sensors, humidity sensors, motion sensors (e.g., accelerometers), applied force sensors (e.g., strain gauges, etc.), location sensors (e.g., GPS sensor), other temperature sensors (e.g., thermistors), or any other sensors, or any combination of the foregoing for obtaining measurements the physical parameters desired by the operator for a particular application.

Power source 121 may include any one or more power sources, including any known in the art or hereafter developed. For example, such power sources may include, but are not limited to, a rechargeable battery, a non-rechargeable battery, a removable battery, a solar cell, motion power conversion circuit, etc.

Processing engine 122 may include any one or more processing engines, including any known in the art or hereafter developed. For example, such processing engines may include, but are not limited to: processors, microprocessors, controllers, microcontrollers, control logic, etc., or any combination of one or more of the foregoing.

Memory 123 may include any one or more volatile or nonvolatile storage units, including but not limited to: ROM, RAM, flash storage, etc., or any combination of one or more of the foregoing.

Communications interface 124 may include any one or more wired or wireless communications transmitters, receivers, transceivers, circuits or communications modules, including any known in the art or hereafter developed or any combination of any one or more of the foregoing. Such communications interfaces 124 may include, but are not limited to: Zigbee communications modules, Bluetooth communications modules, Wi-Fi communications modules, cellular communications modules, RF communications modules, etc., or any combination of one or more of the foregoing.

Audio source 126 may include any one or more audio sources, including any known in the art or hereafter developed. Such audio sources may include, but are not limited to: a speaker.

Light source 128 may include any one or more light sources, including any known in the art or hereafter developed. Such light sources may include, but are not limited to: LEDs, lasers, incandescent light sources, etc., or any combination of one or more of the foregoing.

Other components 125 may include any other electronic component desired to be integrated with the HMD 100 to implement system 1000. For example, other components 125 may include additional or alternative sensors, antennae, or other circuitry. In some embodiments, other components 125 may include an electronic ID such as an Electronic Serial Number (ESN), and ID tag, and ID chip, or other componentry to provide a unique identifier for the HMD 100. Such electronic ID may be actively incorporated into data packets transmitted from the communications interface 124, or may be passively accessible to other computing devices requesting ID information from the particular HMD 100. Such an electronic ID may be associated with a particular animal (for example, based on the animal's ID number (“AID”), the animal's ear tag ID number (“TID”), or both where the AID is different from the TID (just like a human's social security number is different than their driver's license number, the animal's AID number may be different than its TID number). The association between an HMD 100, an animal's AID, and/or an animal's TID may be established automatically or manually (e.g., manually entered by an administrator or other user of system 1000) to provide receivers 200, computing platforms 300, and/or client computing devices 400 with added source information to effectively and efficiently aid in sorting, organizing, storing, or otherwise processing information transmitted from a given HMD 100.

Each of the elements depicted in FIG. 5 may be operatively coupled together (e.g., via bus 129) to effectuate one or more of the features provided by the present disclosure. For example, thermistor 116 may be operatively coupled with processing engine 122, memory 123, and communications interface 124, for example. Memory 123 may be configured with machine readable instructions which are executed periodically (e.g., on a loop every fifteen minutes, for example), and when executed cause processing engine 122 to process a signal transduced or otherwise provided via thermistor 116 (and the related thermistor circuitry, e.g., wires in cord 114, sensor circuitry 127, etc.). The processing engine 122 may, based on machine readable instructions stored in memory 123, cause a discretized data packet to be created from analog electrical signal transduced, and cause the discretized data packet to be transmitted via communications interface 124 (and optionally stored in memory 123 or an electronic storage) in accordance with an appropriate communications interface protocol (e.g., Zigbee, Bluetooth, Wi-Fi etc.).

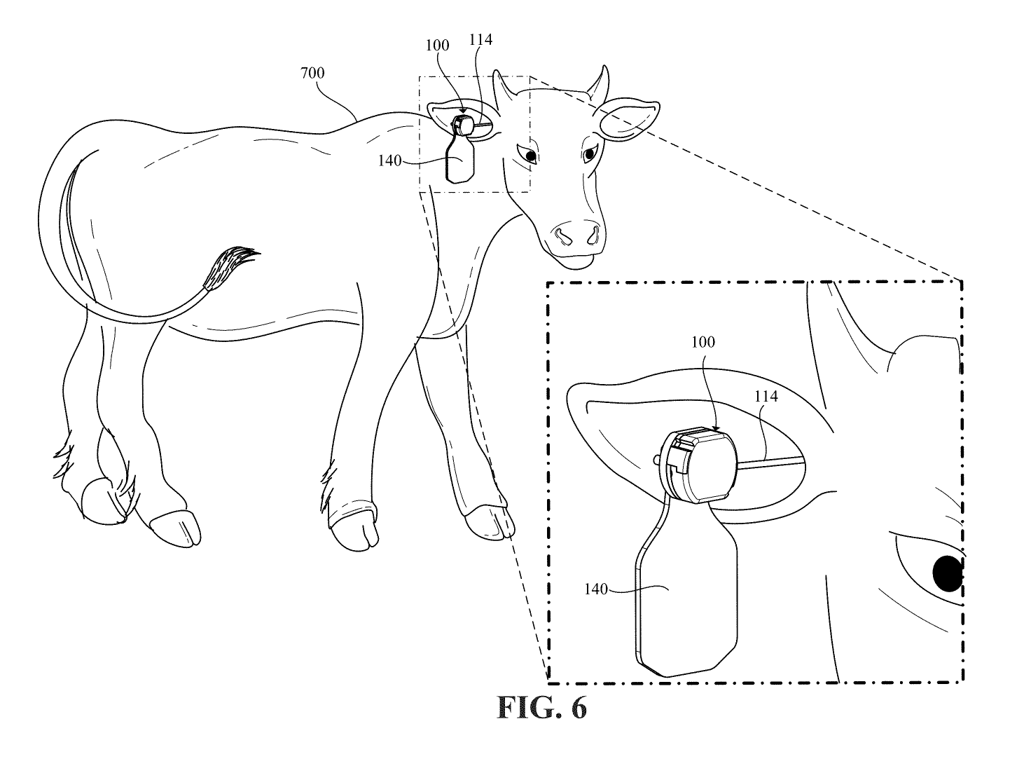

FIG. 6 illustrates an example HMD 100 installed on an animal, in this depiction attached to an ear of a cow 700 in accordance with one or more embodiments of the present disclosure. As shown, in some embodiments an HMD 100 may be attached to an animal's ear by piercing the studs 108 of HMD 100 through the scapha or antihelix region of the animal's ear, for example. Cord 114 may extend into the animal's ear such that thermistor 116 is disposed in the animal's ear canal to obtain desired temperature readings (if such readings are desired for the given application). As shown, ear tag identifier 140 associated with the animal 700 may be deployed with the HMD 100 of the present disclosure. The ear tag identifier 140 include a number (e.g., a TID number) may be associated with not only the animal of interest (e.g., associated with the animal's AID), but also associated with an electronic ID of the HMD 100 installed on the animal. In some embodiments, an HMD 100 assigned to one animal may later be reassigned to another animal. For instance, if an animal dies but the operator wants to reuse the deceased animal's HMD 100 to monitor the health of another animal, the operator may reassign the HMD 100 to the new animal (e.g., within system 1000, associate the electronic ID of the HMD with either or both of the AID of the new animal and/or the TID on the ear tag identifier 140 to be used with the new animal). It should be understood that the EID's of the HMDs, as well as the AID's of the animals and the TID's of the ear tag identifiers may be swapped, reassigned, or otherwise changed as needed in a particular operation. As noted previously, associating the right animal with the right HMD 100 may be important for the applications of the technology disclosed herein, depending on the application and needs of the operator. This will become even more clear as other applications and functionality are discussed herein.

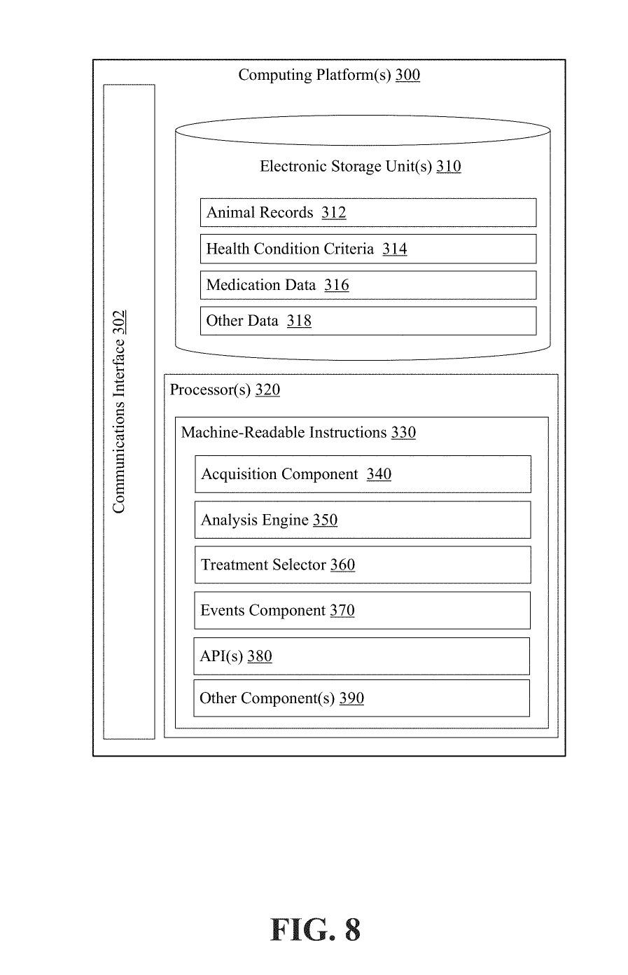

Referring back now to FIG. 1, HMD 100 of system 1000 may be communicatively coupled to one or more receivers 200 and/or computing platforms 300 and/or client computing devices 400. Client computing devices 400 may include a personal computer, a mobile smartphone, a netbook, a tablet or any other computing device capable of connecting to the computing platform over a communications link 900. Computing platforms 300 may receive information from one or more of the other elements of system 1000, make determinations based on that information, and make such information and determinations accessible to client computing devices 400 or other portals, discussed in more detail with respect to FIG. 8. Before the detailed discussion of FIG. 8, however, it is illustrative to describe an example environment within which system 1000 may be used.

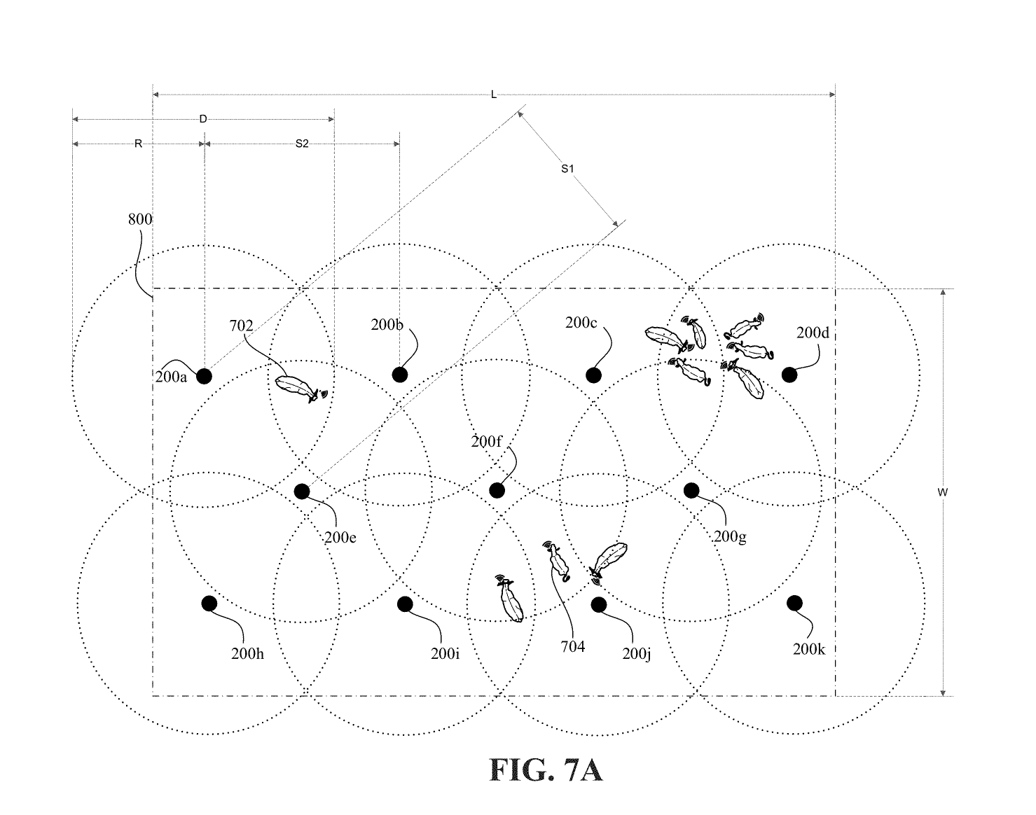

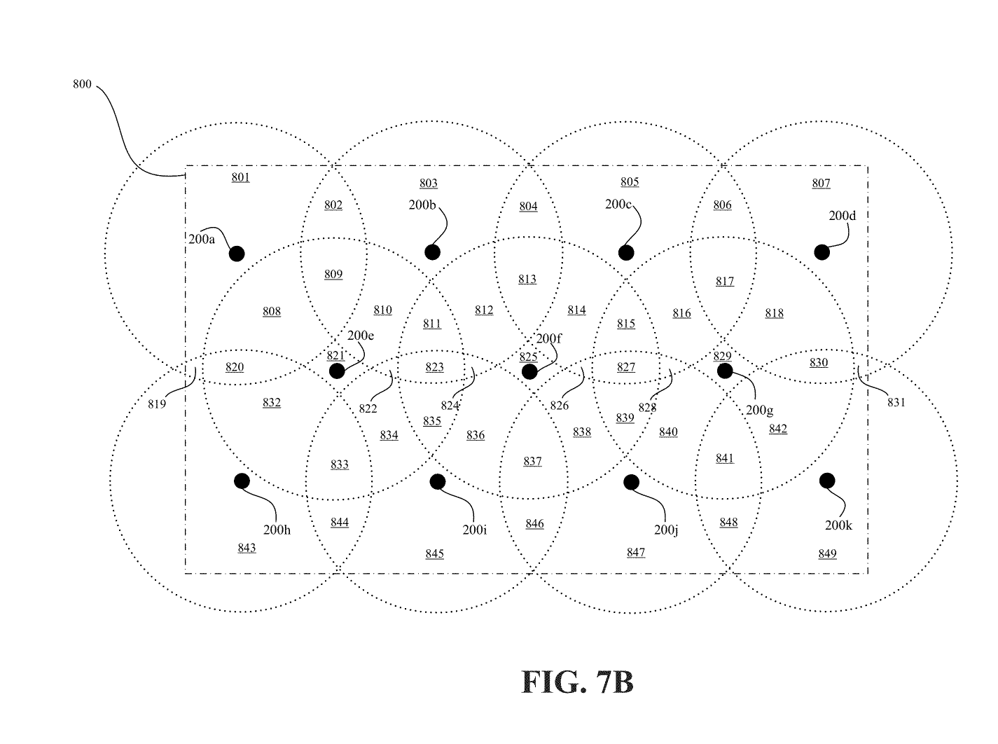

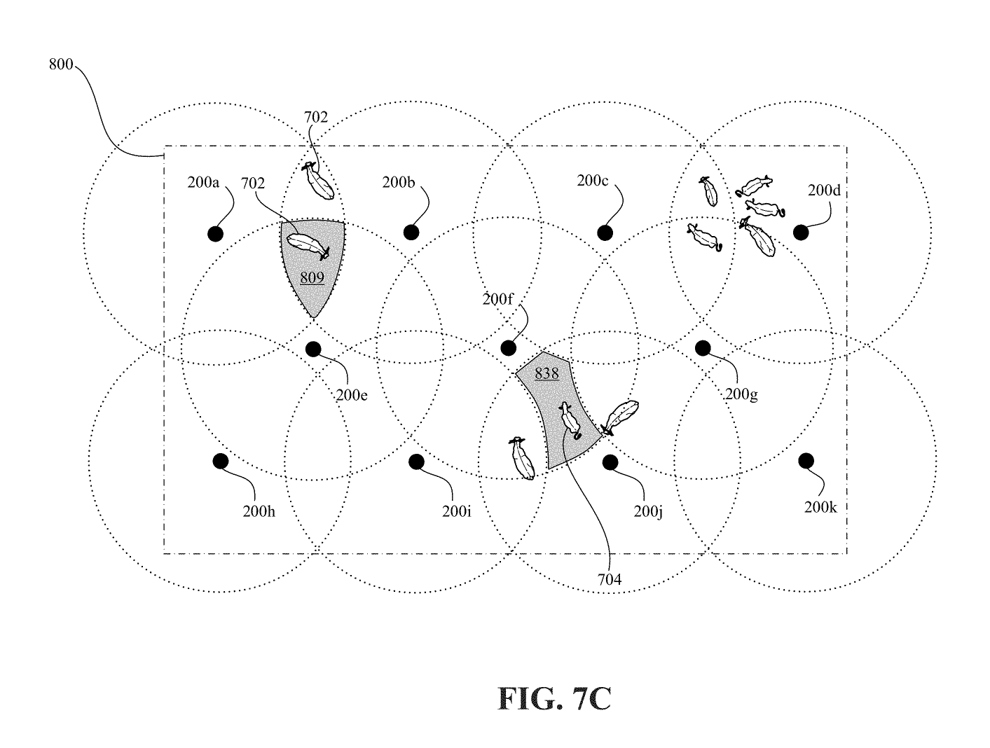

FIGS. 7A-7C illustrate an example environment within which one or more embodiments of the systems, methods, and apparatus of the present disclosure may be implemented. As these figures depict essentially the same example environment, they will be discussed together with like numerals referring to like elements.

As shown in FIG. 7A, the example environment includes a field 800 containing ten cattle, each with an example HMD 100 installed in their ear, each example HMD 100 having a thermistor disposed within the ear canal of the respective cow to which it is installed. Placed at various locations throughout the field are a plurality of receivers 200, namely: receiver 200a, receiver 200b, receiver 200c, receiver 200d, receiver 200e, receiver 200f, receiver 200g, receiver 200h, receiver 200i, receiver 200j, and receiver 200k. In the depicted example, the dashed-line circles around each receiver are a symbolic representation of a successful communication zone for communication with the respective receiver, e.g., the maximum distance away from each receiver within which signal transmissions to/from an HMD 100 may be successful.

As shown, R may represent the maximum distance from a receiver for which successful signal communications with an HMD 100 may occur. That is, when an HMD 100 is more than a distance R from a given receiver, the signal transmitted by the HMD 100 will not (or is highly unlikely to) reach the receiver. In configurations where the receivers are configured to poll the HMDs for updates (e.g., the receivers 200 actively transmitting or broadcasting an information request via a transmitter coupled with the receiver), HMD 100's located beyond a distance R from the polling receiver will not (or are highly unlikely to) receive the transmission. One of ordinary skill in the art will appreciate that the distance R may be limited or defined by the power of the signals that can be generated by the transmitter of the communications interface 124 at the HMDs 100, the power of the signals that can be generated by the transmitter of the communications interface of the relevant receiver 200 (or other communications node, or even the computing platform 300 where the computing platform has a communications interface configured to be communicatively coupled with an HMD 100), or both.

As shown, D may represent the diameter of the circle drawn around each receiver, based on the distance R, delineating the successful communications zone, e.g., the zone within which successful communications between the given receiver and a given HMD 100 may take place. As shown, S1 may represent the distance between a receiver and its nearest neighbor in the receiver network (as depicted in FIG. 7A, S1 is the distance between receiver 200a and receiver 200e), and S2 may represent the distance between a given receiver and its second nearest neighbor (as depicted in FIG. 7A, S2 is the distance between receiver 200a and receiver 200b). In the depicted environment shown in FIG. 7A, S1<S2 by a small margin. This illustrates that in some embodiments the distribution of receivers in a given environment need not be perfectly uniform for system 1000 to operate. While in some embodiments the receiver network may include a substantially uniform distribution of receivers (S1=S2), in other embodiments the receiver network may include a non-uniform distribution of receivers (S1≠S2).

The arrangement of receivers deployed with system 1000 may take any form, e.g., a grid pattern, a hatch pattern, staggered pattern, etc. In some embodiments, it may be desirable to arrange the position of the receivers 200 within the network such that the successful communication zones overlap enough that all or substantially all the geographic area where the animals are roaming will fall within the successful communication zone of at least one receiver 200. Some embodiments may call for less complete coverage, and others more robust coverage, depending on the requirements of the operation.

It is well known that electromagnetic signals become more attenuated with increased distance of the transmission, and the increased impurities in the medium through which the signal must travel. In many applications, such as the one shown in FIGS. 7A-7C, the medium (or the channel through which the transmitted signal travels) is outdoor air. Receiver arrangements configured for increased overlap in successful communication zones will generally yield an increasingly robust communications environment (i.e., signal transmissions will occur with increasing success). The receiver 200 arrangement may be adjusted according to the needs and resources of the operation, in accordance with one or more embodiments of the present disclosure. Indeed, the present technology may be implemented in any customizable manner that meets the requirements and objectives of the relevant animal caregiver or operation generally.

Overlapping successful communication zones can result in situations where an HMD 100 is within range of multiple receivers when transmitting a signal. In some embodiments, multiple receivers may receive the signal transmitted by the communications interface 124 of an HMD 100. As described in more detail with reference to the events component 370 of computing platforms 300, system 1000 may utilize such an arrangement to provide enhanced location features to operators and other animal caregivers. In some embodiments, system 1000 may utilize information available to computing platforms 300 to compute or otherwise derive (e.g., via trilateration, triangulation, or any other location computation), with varying degrees of accuracy depending on the arrangement of receivers 200, the location of the animal associated with an HMD 100 whose signal transmission was detected by multiple receivers 200.

For example, referring now to FIG. 7B, suppose field 800 were divided into a plurality of subzones defined by the boundaries of neighboring receivers and/or the boundary of the field 800. As shown in FIG. 7B, each zone is represented by a numeral in the 800 range. For example, the boundaries of subzone 801 are delineated by the upper left corner boundary lines of the field 800, as well as the dotted lines of the overlapping success zone lines of receiver 200b, receiver 200e, and receiver 200h. Subzones 802 through 849 are also shown, for reference.

Turning now to FIG. 7C, suppose for example that a herd of cattle are roaming in field 800. Suppose further that a scheduled signal transmission occurs at 12:00 pm wherein each cow's attached HMD 100 transmits a signal (e.g., enveloping data) including the temperature information obtained via each HMD 100's respective thermistor. In some embodiments, signals containing information about each cow's temperatures are received by one or more receivers (depending on overlap), and thereafter provided to computing platforms 300 or other elements of system 1000. System 1000 may obtain the temperature information and, based on one or more health condition criteria 314 (discussed in more detail below), determine that: (i) the temperature reading(s) associated with one or more cows indicate the onset of a treatable health condition (e.g., cow 702 and cow 704 as shown in FIG. 7C, for example)), and (ii) may further determine that a certain treatment modality should be delivered to such cows (e.g., an antibiotic should be delivered to cow 702 and cow 704 within the next five hours), and (iii) may notify the animal's handler accordingly (e.g., via text, call, email, mobile application alert or notification to cow 702 and cow 704's handler, etc.), among other things, as discussed in more detail below.

In some embodiments, automatically or upon request by the animal's handler (via mobile application, text message, internet, or otherwise) system 1000 may determine and/or provide location information associated with one or more animals within the field 800 that need to be pulled (e.g., animals needing treatment).

For example, system 1000 may determine that the 12:00 pm transmission by cow 702's HMD 100 was picked up by receiver 200a, receiver 200b and receiver 200e. Based on this information, as well as the zone and subzone region information, system 1000 may determine that cow 702 must be in subzone 809, the only region where all three receivers could have successfully received a transmission from cow 702's HMD 100. Similarly, system 1000 may determine that the 12:00 pm transmission by cow 704's HMD 100 was picked up by receiver 200j and receiver 200f. Based on this information, as well as the zone and/or subzone region information, system 1000 may determine that cow 704 must be in subzone 838, the only region where both of receiver 200f and receiver 200j could have successfully received the transmission from cow 704's HMD 100 without also being picked up by another receiver.

Accordingly, in some embodiments system 1000 may determine an approximate location for an animal of interest based on which receivers picked up (e.g., received) the relevant HMD 100's signal transmission (or vice versa for the polling configuration). In other embodiments, HMD 100 may include a GPS module to provide location information. Thus, there are various ways that system 1000 may determine and provide location information about a given animal. Such information may be further utilized by system 1000 to provide directions to a caregiver, such as a handler, to find the relevant animal. System 1000 may provide directions as an audible or visual description, and audible or visual instruction, or a visual diagram (e.g., via a route mapping application, or the like) on a client computing device 400. The directions may include step-by-step instructions or a visual route displayed on the handler's client computing device 400 (e.g., the handler's iPhone or other smartphone), and may be based on the handler's current location as provided via a GPS module in the handler's smartphone (which system 1000 may be configured to access).

As noted previously, HMDs 100 and receivers 200 may be communicatively coupled. That is, they may be equipped with the hardware and software necessary to communicate with one another (e.g., via their respective communications interfaces in accordance with one or more communications protocols known to each). HMDs 100 or receivers 200 or both may further be communicatively coupled, directly or indirectly, with one or more of computing platforms 300 and client computing devices 400. Each element of system 1000 may be equipped with the hardware and software necessary to communicate with one another (e.g., via their respective communications interfaces in accordance with one or more communications protocols known to each).

As indicated above, a user may access one or more pieces of information stored by, determined by, or otherwise accessible through one or more other elements of system 1000. In some embodiments client computing device 400 may provide a portal through which access to computing platform 300 may be provided. For instance, a client computing device 400 may include a desktop computer with a web browser. The web browser may be used to access the computing platform 300 over a wired or wireless internet connection. The user may log in to a portal through an web browser that provides a user interface such as an interactive dashboard including various options and features for consuming, interpreting, modifying and otherwise interacting with the information available through system 1000 (e.g., information on computing platform 300), and with other users associated with the information available through system 1000. The computing platforms 300 may include a server, server network, a database, cloud computing resources, or other computing resources. The computing platform 300 may run one or more APIs enabling communication and/or interactivity between the web based user interface and the computing platform 300.

In another embodiment, a client computing device 400 may include a smartphone running an application providing a user interface (hereinafter, referred to generally as an Animal Management Application, or AMM App). The AMM App may be used to access the computing platform 300 over a wired or wireless internet connection. The user may log in to the AMM App which provides a user interface that may include a dashboard including various options and features for consuming, interpreting, modifying and interacting with the information available through system 1000, and with other users associated with the information available through system 1000. The computing platforms 300 may include a server, server network, a database, cloud computing resources, or other computing resources. The computing platform 300 may run one or more APIs enabling communication and/or interactivity between the AMM App user interface and the computing platform 300.

FIG. 8 illustrates an example computing platform 300 that may be implemented in accordance with one or more embodiments of the present disclosure. Computing platform 300 may take the form of a server, a server farm, a database, a cloud computing platform, and/or any other computing platform. As shown, computing platform(s) 300 may include one or more electronic storage unit(s) 310 to store data and/or operating instructions, one or more physical processor(s) 320, and one or more machine-readable instruction(s) 330 that may be executed by the one or more physical processors 320. In some embodiments, electronic storage units 310 may store data including, for example as shown in FIG. 1, animal records 312, health condition criteria 314, and medication data 316, among other data 318. As shown in FIG. 8, physical processor(s) 320 may be configured by machine-readable instructions 330 to include one or more components that, when executed by physical processor(s) 320, cause or enable computing platform(s) 300 to effectuate one or more of the features described herein.

Such components may include one or more of an acquisition component 340, an analysis engine 350, a treatment selector 360, events component 370, and an Application Programming Interface 380 (“API 380”), among other components 390. As described herein, any of the components or subcomponents described herein may be in operative communication with one another, as well as with any other element or sub element of the system 1000 (e.g., electronic storage unit(s) 310, client computing devices 400, external resources 600, etc.), to effectuate one or more embodiments of the technology disclosed herein.

As shown, electronic storage units 310 may include animal records 312, among other data and records. Animal records 312 may include any static or dynamic information about a specific animal, about a subset of animals within a herd, about a herd generally, or about multiple herds. Such information may include: genetic information (e.g., genus, species, family, subfamily, lineage, breed, gender, ancestry, genetic mutations (albinism, etc.)), medical information (e.g., birth date/place, vaccination history, illness history, injury history, allergies, health susceptibilities, failed or successful impregnation records, failed or successful birthing records, current and past diseases exposed to or sustained, current and past medical operations undergone or anticipated, current and past medical treatment modalities (e.g., current and past treatment regimens, etc.)), phenotype information or physical characteristics (pigmentation (e.g., of fur, hair, skin, eyes, etc.), height, weight, girth, physical deformities (blindness, dwarfism, protoporphyria, heart condition, respiratory condition, etc.)), geographic information (e.g., birthplace; travel history (city, state, zip, farm, ranch, plot, or other locations where the animal was maintained or purchased from (e.g., sometime referred to as the “source,” meaning the place where the animal came from), including length of stay in each place, and any other information about such locations (e.g., elevation, altitude, season of year at one or more times relevant to the animal (e.g., the time or season of the year when the animal was purchased, transferred or otherwise moved from one location to another—i.e., fall, winter, spring, summer, April, May, June, etc.); temperature information (e.g., an animal's detected body temperature obtained at one or more times in the past, external environmental temperature at one or more times in the past where animal was located); altitude or elevation information (e.g., altitude or pressure measurements obtained at one or more times in the past where animal was located); feed/diet information (e.g., feed frequency, feed type, feed allotment, feed times, food allergies, etc.), activity information (e.g., daily exercise regimen (for a racehorse, for example)), production information (e.g., amount of milk a milk cow produced in a given time period, rates of production, etc.), or any other information that can be associated with the animal including financial information (e.g., price paid for animal, costs incurred in connection with animal, revenue/profits generated from animal to date, expected revenue/profits, etc.), responsible caregiver information (e.g., responsible ranch handler's name, number, email address (or other contact or personal information), shift hours, scheduled time off, sick days (or other work related information)), or any other customized predefined metrics computable from physical parameters measured about the animal (e.g., lung score, heart score, speed score, sleep score, production score).

It should be noted that animal record 312 may be manually entered information (e.g., an end user entering information into a data field via a client computing device 400, the information being received by and stored at computing platforms 300), automatically obtained information (e.g., biometric information detected via HMD 100 about a particular animal may be automatically communicated to computing platform 300 and updated in the corresponding animal record 312 stored in electronic storage unit(s) 310), automatically generated information (e.g., information based on or derived from any entered or automatically obtained information), or any combination of the foregoing or other desirable manner (e.g., an end user may select, via their client computing device 400, an option to sync the computing platform 300 with an external resource 600 such that the computing platform 300 is updated with information from, for example, an online database accessible over an Internet connection).

For example, an animal record for a cow on a farm may include an entry in the animal record for the cow's date of birth and the cow's current age. The date of birth may be manually entered to a newly created animal record once the cow is born. The cow's age may be automatically updated as computing platform 300 automatically obtains current date information (e.g., from an external resource such as an online clock or calendar) and automatically generates or updates an entry for the cow's age based on the difference in the current date obtained and the cow's entered date of birth. One of skill in the art will appreciate that the animal record 312 may include both static information, or in other words unchanging information (e.g., animal date of birth), and active or dynamic information, information that is subject to change based on other occurrences (e.g., animal age based on the passage of time). Additionally, one of skill in the art will appreciate that the information maintained in animal records 312 may be configured such that it is as condensed or extensive as desired by the end user (e.g., as desired by the client, the owner, operator, manager, administrator, supervisor, handler, the farmer, the ranch operation, an employee, etc.).

Animal records 312, including medical histories of one or more animals, may be stored securely within system 1000 (e.g., utilizing digital signatures or cryptographic protection) to prevent information in animal records from becoming compromised, or reduce the likelihood of the same.

As further shown in FIG. 8, electronic storage unit(s) 310 may include health condition criteria 314, among other data and records. Health condition criteria 314 may include relationships (e.g., rules) between animal categories, animal parameters, and health conditions. In some embodiments, such relationships permit system 1000 to determine or identify the potential onset of a health condition in a particular animal based on the animal's animal category and animal parameters.

Animal categories provide a way to categorize an animal based on one or more factors relevant to its health. Animal categories may be defined as broadly or narrowly as desired for a given application. For example, an animal category may be defined by a genus (e.g. Bos), or a subfamily within a genus (e.g., Bovinae), or a member group within a subfamily, (e.g., Cattle). In some embodiments, the animal category may be defined with much more specificity. For example, an animal category may be defined as: male cattle with black and white spotted hides between the ages of 6 and 9 and weighing between 2000 and 2500 pounds. A person of ordinary skill in the art will appreciate upon reading this disclosure that animal categories can be defined with as much granularity as desired for a given application, including with specificity as to any of the various types of information discussed herein with respect to animal records, among other information that might be relevant to an animal's health. The present disclosure is intended to extend to all such animal categories, defined in any manner desired for a given application.

Animal parameters include any physical attribute or condition about an animal, or about an animal's environment, that may be quantifiably measured. For instance, an animal parameter may include the animal's core temperature, heart rate, perspiration or salivation levels, activity (e.g., speed), geographic location (e.g., GPS coordinates, altitude, etc.), ambient temperature in the environment where the animal is located, wind-chill factor in the environment where the animal is located, etc.

Health conditions include any condition that describes the state of a structure or function of a living organism's body. Health conditions can include any type of disease, illness, symptom, injury, mutation, or otherwise, whether ongoing, chronic, temporary or permanent. To illustrate an example of how health condition criteria may, in some embodiments, define relationships between animal categories, animal parameters, and health conditions, consider the following form of an exemplary health condition criteria: for [animal category], a [animal parameter] exceeding [range] for [time period], is associated with [health condition]. For example: for cattle having entirely black hides, a core body temperature exceeding 102.5 degrees Fahrenheit for more than 1 hour is associated with Bovine Respiratory Disease Complex (BRDC). Thus, when system 1000 obtains core body temperature readings computed from information obtained from a cow's HMD that satisfies the criteria, system 1000 may determine that the given cow could have BRCD. Of course, as one of ordinary skill in the art will appreciate, the health condition criteria may be defined in any manner, for any disease, with as much granularity as desired or as understood for a particular animal and/or animal category. The health condition criteria may be readily defined in software and/or stored in hardware.

As described in more detail below, health condition criteria, if satisfied, may indicate the animal associated with the measured attribute is developing (or has already developed) the associated health condition and may need some form of treatment.

As further shown in FIG. 8, electronic storage unit(s) 310 may include medication data 316, among other data and records. Medication data 316 may include static or dynamic information about medications that may be applied or provided to an animal with a given health condition. Such static or dynamic information can be pricing information about a given medication (e.g., price paid for the medication, current market price to purchase more, etc.), availability information (e.g., amount of the given medication currently in stock, amount of the given medication available from a particular seller or group of sellers, contact or website information through which more of a given medication may be purchased, delivery times or estimated delivery dates for orders of a given drug, etc.), effectiveness (e.g., effectiveness for treating a given health condition generally, effectiveness for treating a given health condition for an animal of a particular animal category, effectiveness for treating a given health condition for an animal of a particular animal category with a given measure of animal parameters), etc. Medication data 316 may be entered manually (e.g., end user manually inputting updates about the stock of medication maintained at the farm after the end user has used up some of the stock of the drug by treating an animal with a health condition) or may be updated automatically (e.g., receiving updates to dynamic information, such as current market pricing, updated research findings/warnings about a given drug, or newly discovered symptoms or side effects for certain drugs, updated instructions for delivery of a given medication that may be obtained from an external resource 600 such as an online database).

As further shown in FIG. 8, computing platform(s) 300 may include one or more physical processor(s) 320 configured with machine-readable instruction(s) 330 which, when executed, effectuate one or more of the features of the present disclosure. In some embodiments, machine-readable instruction(s) 330 may include one or more of an acquisition component 340, an analysis engine 350, a treatment selector 360, an events component 370, an API 380, among other components 390.

Acquisition component 340 obtains data representing physical measurements (the animal parameters) detected and transduced by HMD 100. Acquisition component 332 may be operatively coupled with a communications interface 302 of computing platform 330 that receives signals representing the data, either directly or indirectly, from an HMD 100. Acquisition component 340 may obtain the data enveloped by the signals periodically, on an automated basis, or any given time upon a user-initiated request. Acquisition component 340 may associate the animal parameter data, permanently or temporarily, with the animal record 340 stored in electronic storage units 310. Optionally, acquisition component 340 may determine whether the physical measurement was accurately obtained.

In one example, acquisition component 340 may determine whether the physical measurements fall within a realistic range. For instance, a range of realistic body temperatures for a given type of cattle may be stored in electronic storage 310 as being between 70 and 100 degrees Fahrenheit, and when acquisition component 340 obtains data representing a given cow's body temperature of 200 degrees Fahrenheit, Acquisition component 340 may determine that the measured temperature is outside the realistic range, and filter out or otherwise discard the data as null.

In another example, the acquisition component 332 may determine whether the HMD 100 is operating properly (based on HW status component 395, for example, as discussed below). If acquisition component 340 determines that the physical measurement was obtained during a time when the HMD was not working properly, or was otherwise malfunctioning, acquisition component 340 may filter out or otherwise discard the data as null.

Analysis engine 350 determines whether one or more health condition criteria 314 have been satisfied for a given animal being monitored by HMD 100, based on the data obtained by acquisition component 340. Extending the example above—where the health condition criteria was described as: for cattle having entirely black hides, a core body temperature exceeding 102.5 degrees Fahrenheit for more than 1 hour is associated with Bovine Respiratory Disease Complex (BRDC)—suppose that one of a farmers entirely black hided cattle has an ear tag numbered 702 which has been associated with a HMD 100 installed on the cow's ear. Suppose also that the HMD 100 measures cow 702's core temperature and transmits a signal representative of that temperature on a periodic basis, e.g., every 15 minutes, for reception by a receiver and relay to computing platform 330. Either through direct or indirect reception, acquisition component 340 may acquire the temperature information that is obtained by the respective HMD 100 every 15 minutes, for example, and may optionally facilitate storage of the information in the animal record 312 associated with cow 702 in one or more electronic storage unit(s) 310. Analysis engine 350 may monitor the temperature readings obtained by acquisition component 340, evaluate those temperature readings with respect to the health condition criteria 314, and determine whether one or more health condition criteria 314 have been satisfied. Again, referring to the example above, suppose that the following measurements in Table 1 below have been obtained from HMD 100 installed on cow 702's ear over the course of a four-hour period:

TABLE 1

| | ANIMAL RECORD_AID_TID 702_TEMPERATURE DATA | | | | | |
|---|---|---|---|---|---|---|
| | A | B | C | D | E | F |
| 1 | Date | Time | Core Temp. | Ambient | . . . | . . . |
| 2 | Aug. 26, 2017 | 12:00 | 101.5° F. | 78° F. | | |
| 3 | Aug. 26, 2017 | 12:15 | 101.5° F. | 78° F. | | |
| 4 | Aug. 26, 2017 | 12:30 | 102° F. | 78° F. | | |
| 5 | Aug. 26, 2017 | 12:45 | 102.5° F. | 78° F. | | |
| 6 | Aug. 26, 2017 | 1:00 PM | 102.5° F. | 79° F. | | |
| 7 | Aug. 26, 2017 | 1:15 PM | 102.7° F. | 79° F. | | |
| 8 | Aug. 26, 2017 | 1:30 PM | 102.7° F. | 79° F. | | |
| 9 | Aug. 26, 2017 | 1:45 PM | 102.7° F. | 78° F. | | |
| 10 | Aug. 26, 2017 | 2:00 PM | 102.9° F. | 78° F. | | |
| 11 | Aug. 26, 2017 | 2:15 PM | 102.9° F. | 78° F. | | |
| 12 | Aug. 26, 2017 | 2:30 PM | 103° F. | 78° F. | | |
| 13 | Aug. 26, 2017 | 2:45 PM | 102.8° F. | 77° F. | | |
| 14 | Aug. 26, 2017 | 3:00 PM | 102.8° F. | 77° F. | | |
| 15 | Aug. 26, 2017 | 3:15 PM | 102.7° F. | 77° F. | | |
| 16 | Aug. 26, 2017 | 3:30 PM | 102.8° F. | 77° F. | | |
| 17 | Aug. 26, 2017 | 3:45 PM | 102.9° F. | 77° F. | | |
| 18 | Aug. 26, 2017 | 4:00 PM | 102.7° F. | 77 F. | | |

As may be seen from the table of temperature data for cow 702 above, core temperature readings from the HMD 100 begin to exceed the temperature threshold of 102.5° F. (defined by the health condition criteria) at approximately 1:15 pm on Aug. 26, 2017 and continues to increase without ever falling back to 102.5° F. or below. Based on the predefined health condition criteria 314, analysis engine 350 may determine that health condition criteria has been satisfied for the BRDC disease as of at least 2:30 pm (more than 1 hour spent over the 102.5° F. threshold). In other words, analysis engine 350 evaluates the information obtained from HMD 100 (via acquisition component 340, or otherwise) and generates a diagnosis (or preliminary diagnosis, depending on certainty levels accorded to the given health condition criteria), if any, of a health condition that the respective animal may be developing (or have already developed) based on the predefined health condition criteria 314.

It should be appreciated by those of skill in the art that the scenario described above with respect to cow 702 is a descriptive example provided for illustration purposes, and is in no way intended to be limiting of the scope of the present disclosure. A person of skill in the art should appreciate upon reading this disclosure that the health condition criteria 314 may be defined as simply (e.g., a simple threshold based rule) or complex (e.g., based on patterns of data over a given timeframe, based on multiple interdependent parameters that—taken together—satisfy a rule) as desired. Just as the health condition criteria 314 can be as granular or detailed as desired, so to can the analysis engine 350's evaluation of the information available to it, which may inform or provide a preliminary or ultimate diagnosis for a given animal.

Once analysis engine 350 has determined that one or more health condition criteria 314 have been satisfied for a given animal, treatment selector 360 determines which treatment modality among one or more treatment modalities to apply (or recommend be applied) to the given animal under the circumstances. There may be many treatment options available for the identified health condition for a certain category of animal. Treatment selector 360 may consider one or more factors, drawing from one or more pieces of information accessible to system 1000 in identifying an appropriate treatment modality under the circumstances. The various pieces of information that treatment selector 360 may consider include any and all of the information accessible to system 1000. In some embodiments treatment selector 360 may determine which of two or more treatment modalities should be applied based on one or more factors and information available to system 1000 that are relevant to those factors.

For example, a factor considered by treatment selector 360 may be “age.” The one or more pieces of information relevant to that factor and accessible to system 1000 may include the date of birth for the given animal under consideration (e.g., obtained from animal records 312, obtained from an estimate provided by an operator, obtained from another resource, etc.) and the present date (e.g., obtained from an electronic clock of computing platform 300, or obtained from an external resource 600 such as an online calendar operably connected thereto, etc.). Treatment selector 360 may determine how old the given animal is by determining the temporal difference between the current date and the date of birth for the animal. Treatment selector 360 determine appropriate treatment modalities in view of the animals age, for example. For instance, if the given animal is under a particular age (e.g., two years old), treatment selector 360 may determine that antibiotic A is a better alternative than antibiotic B under the circumstances because information about antibiotic B (which may be stored in medication data 316, or available through an external resource 600) indicates there are harmful side-effects when antibiotic B is given to animals under the age of two.

In some embodiments treatment selector 360 may rank competing treatment modalities based on one or more factors and the information accessible to it that are relevant to those factors. In some embodiments the factors that treatment selector 360 may consider can be defined, in whole or in part, by default. In some embodiments the factors that treatment selector 360 may consider can be defined, in whole or in part, by an end user (e.g., a farmer who has adopted system 1000 for monitoring his livestock). The following three examples help illustrate just a few of the factors and related information treatment selector 360 may assess in its determination of one or more treatment modalities.

As a first example, suppose that analysis engine 350 has determined that cow 702 has developed a health condition, namely Bacterial Infection X. Treatment Selector 360 may draw on information within medication data 316 to determine which medications are designated for use to treat Bacterial Infection X in cattle. Treatment Selector 360 may identify six different antibiotics that are appropriate for treating Bacterial Infection X in cattle. Treatment Selector may obtain information from animal records 312 that is associated with cow 702 in order to assess factors that weigh for or against one or more of the six different antibiotics. For instance, animal records 312 for cow 702 may include a medical report indicating that cow 702 has a fatal allergy to an active ingredient in antibiotic 1 and antibiotic 3, and a non-fatal allergy to an inactive ingredient in antibiotic 2, leaving antibiotics 4, 5, and 6 as the only options that are not known to cause an allergic reaction in cow 702.

Treatment Selector 360 may further obtain information from medication data 316 to determine how quickly the animal should be treated for best results. For example, treatment selector 360 may obtain information from medication data 316 indicating that, absent an antibiotic, Bacterial Infection X is highly contagious in the first 24 hours after onset. Treatment selector 360 may obtain the size of the herd cow 702 is a member of based on the animal records 312 data, and determine that cow should be given an antibiotic within the next two hours. Treatment selector 360 may draw again on information from medication data 316 (or other data 318) to determine which of the antibiotics are currently in stock at the farm (such that they could reasonably be given to the animal within the two-hour timeframe). Treatment selector 360 may determine that antibiotics 3, 4 and 6 are currently in stock at the farm, antibiotic 1 is out of stock at the farm but can be brought to the farm by a courier at a local vendor within 5 hours, antibiotic 2 is out of stock and not shippable or deliverable, and antibiotic 5 is out of stock and can be shipped with a guaranteed delivery time of 48 hours.

Treatment selector 360 may further draw on medication data 316 to determine pricing information for the antibiotics. Treatment selector may determine that the cost for the twice a day for five day dosage schedule (i.e., delivery regimen) for antibiotic 1 is $5.00, the cost for the once a day for seven day delivery regimen for antibiotic 2 is $20.00, the cost for the three times a day for two day delivery regimen for antibiotic 3 is $60.00, the cost for the once a day for seven day delivery regimen for antibiotic 4 is $60.00, the cost for the three times a day for two day delivery regimen for antibiotic 5 is $55.00, the cost for a one-time only dose of antibiotic 6 is $65.00. Treatment selector 360 may eliminate antibiotics 1, 2 and 3 on account of cow 702's allergies; may eliminate antibiotic 5 because it is not in stock at the farm and thus cannot meet the imminent delivery timetable to prevent the infection from spreading to other cattle in the herd; may eliminate antibiotic 6 because it is the most expensive, and thus may select antibiotic 4 as the treatment for cow 702. Thus, treatment selector 360 may determine which treatment modality to apply to cow 702 based on a process of elimination approach based on various considerations.

Alternatively or additionally, treatment selector 360 may rank the competing treatment modalities. This may be done in any desired manner, applying any desired priority rules based on the application and the end user's interests. For example, the rank may be based on a weighted score that accounts for various factors of interest to an operator or other user. For instance, consider the following expression in equation 1 which takes the scores for N factors of interest, weights the scores according to a weighting factor, w_{N}, then sums them up to obtain the overall score for a particular treatment modality, MODALITY SCORE.

MODALITY SCORE=(Factor1_{score})w₁+(Factor2_{score})w₂+ . . . +(FactorN_{score})w_{N} [1]

For the given Factors of interest (e.g., allergies, availability, cost, etc.), an initial score, FactorN_{score}, may be given by any formula, which may or may not include further weighting in addition to the weighting factor, w_{N}, applied in equation 1. For example, if allergies were Factor 1, availability of the medication were Factor 2, and cost were Factor 3, an example treatment selector 360 may apply the following:

$$\begin{matrix}{Factor 1}_{score} = {allergies}_{score} = \{ \begin{matrix}0.0 , & fatal allergy \\ 0.5 , & nonfatal allergy \\ 1.0 , & no allergy\end{matrix} & [ 2 ] \\ {Factor2}_{score} = {availability}_{score} = \{ \begin{matrix}0.0 , & Out of Stock ; Not Deliverable \\ 1 / x , & Out of Stock ; Deliverable within [ x ] hours \\ 1.0 , & In Stock\end{matrix} & [ 3 ] \\ {Factor3}_{score} = {cost}_{score} = \frac{1}{[ x ]} * 1.075 , [ x ] = price for medication regimen & [ 4 ]\end{matrix}$$

Extending the examples above, suppose treatment selector 360 accesses various information from system 1000 and applies the example relationships above with respect to each of the six antibiotics under consideration for cow 702. Suppose also, for simplicity, that weighting factors w₁,w₂,w₃=1. The relevant factors, and computed MODALITY SCORE based on those factors may be given as follows in Table 2:

TABLE 2

| | | | | MODALITY |
|---|---|---|---|---|
| | | | | SCORE |
| | Factor 1_{score} | Factor 2_{score} | Factor 3_{score} | Modality |
| | allergies_{score} | availability_{score} | cost_{score} | SCORE |
| Anti- | 0 = 0 | 1/[5] = 0.2 | (1/[$5]) * 1.075 = | 0.4150 |
| biotic | | | 0.2150 | |
| 1 | | | | |
| Anti- | 0.5 | 0 | (1/[$20]) * 1.075 = | 0.5538 |
| biotic | | | 0.0538 | |
| 2 | | | | |
| Anti- | 0 | 1 | (1/[$60]) * 1.075 = | 1.0179 |
| biotic | | | 0.0179 | |
| 3 | | | | |
| Anti- | 1 | 1 | (1/[$60]) * 1.075 = | 2.0179 |
| biotic | | | 0.0179 | |
| 4 | | | | |
| Anti- | 1 | 1/[48] = 0.02 | (1/[$55]) * 1.075 = | 1.0395 |
| biotic | | | 0.0195 | |
| 5 | | | | |
| Anti- | 1 | 1 | (1/[$65]) * 1.075 = | 2.0165 |
| biotic | | | 0.0165 | |
| 6 | | | | |

Thus, treatment selector 360 may, based on their respective MODALITY SCORES, determine a rank of six antibiotics identified as appropriate for treating Bacterial Infection X in cow 702 as:

| | RANK |
|---|---|
| Antibiotic 4 | 1 |
| Antibiotic 6 | 2 |
| Antibiotic 5 | 3 |
| Antibiotic 3 | 4 |
| Antibiotic 2 | 5 |
| Antibiotic 1 | 6 |

As shown in the example table above, treatment selector 360 determined that Antibiotic 4 is the treatment modality most appropriate for cow 702 under the circumstances based on Antibiotic 4 having the highest MODALITY SCORE. Although the example ranking approach and the example elimination approach applied by treatment selector 360 shown above both identified Antibiotic 4 as the most desirable alternative among the existing options, this may not always be the case. Indeed, one of ordinary skill in the art will appreciate that the expressions above are merely exemplary, and any modifications, variations, or other algorithms may be applied to account for the considerations of greatest import for a given application.

For instance, a smaller farming operation may be more interested in the cost of a given treatment modality, and thus may use a different expression to compute a score for that factor (or may apply a much greater weight, w₃, to that factor when computing the MODALITY SCORE), for example. By way of another example, a farmer who doesn't have a large workforce to go around applying the treatment modality to all the sick animals may opt to include a factor that accounts for the number of doses each antibiotic regimen requires, and assign a higher score to those treatment modalities that require the least number of doses.

In another example, the treatment selector 360 may generate a herd-specific effectiveness score. For example, treatment selector 360 may draw on animal records 312 for the herd within which cow 702 resides, and compute a score that represents how effective a given treatment modality has been within the particular herd. This provides farmers with much more robust information relevant to their specific herds. For instance, antibiotic 4 may have previously been given to 7 other cows in the same herd, and in all but one case the cattle died; while antibiotic 6 may have been given to 10 cows in the herd, and in all but one case the cattle returned to full health. Thus, the treatment selector 360 may compute a herd specific effectiveness score for antibiotic 4 of, e.g., 1/7=0.14, and for antibiotic 6 of, e.g., 9/10=0.9, which may be considered in computing the MODALITY SCORE. Extending the example above, if a farmer had also included a factor such as the above herd-specific effectiveness in the computation of the MODALITY SCORE, the ranking procedure would have ranked antibiotic 6 highest, despite it being a more expensive drug than antibiotic 4, for example.

In some embodiments, such data may be collected and shared among farmers in particular regions, or with veterinarians throughout various regions. This way, as between competing treatment modalities, the more effective options may be selected based on relevant prior usage (e.g., use by others in the region with similar animals in the same climate, for instance) that is partially or entirely independent of the underlying physiological properties or historical medical understanding behind each option. Thus, the present technology gives farmers and ranch operators (among others) the flexibility and tools to more carefully and precisely care for their animals, and make more intelligent decisions when treating health conditions. Their decisions can be based on any of the information discussed in the present disclosure.

In some embodiments the treatment selector 360 may provide the results of its determination, or make such available, to client computing devices 400 (e.g., as an update to a website, mobile application, or other interface accessible by the relevant employees or managers involved in farm operation). In some embodiments, treatment selector 360 provides its determinations to an events component 370 (discussed in more detail below) that coordinates task items and/or reporting that aids in ensuring that relevant animals obtain relevant treatments in a timely manner. In some embodiments, treatment selector 360 and/or events component 370 provide treatment modality determinations to designated end users, including operators, managers, supervisors, handlers, other employees, or even associated veterinarians, who can (i) confirm or deny that the determined treatment modality is appropriate (ii) make a change to the treatment modality determined by the treatment selector 360 (e.g., change the selection from antibiotic 4 to antibiotic 5 based on factors not yet known to system 1000 but known to the veterinarian), (iii) prescribe the medication corresponding to the selected/determined treatment modality (if a prescription is necessary), or (iv) leave comment or notes, etc.; any and all of the foregoing can be transmitted back to the computing platform 300, and can optionally be updated/stored in an animal record 312 associated sector of electronic storage 310 or elsewhere on electronic storage 310.

As further shown in FIG. 8, computing platform(s) 300 may include an events component 370. Once analysis engine 350 has determined that one or more health condition criteria 314 have been satisfied for a given animal, and once treatment selector 360 determines which treatment modality to apply to the given animal under the circumstances (which may be changed via user input as described above, e.g., from a veterinarian), events component 370 may generate further requests, updates, notifications, alerts that may be transmitted or otherwise made available to users their client computing devices 400. Events component 370 may further obtain and/or process input received back from users (e.g., input entered via their client computing devices 400). In accordance with such embodiments, events component 370 may coordinate task items, report actions taken, and update system 1000 accordingly to aid in ensuring that relevant animals obtain relevant treatments in a timely fashion, and that relevant updates are made to animal records 312 as relevant events occur.

In some embodiments, events component 370 may obtain determinations made by treatment selector 360 (and optionally confirmed or changed by a designated veterinarian or other operator), and, based on information obtained from electronic storage units 310 about the animal's handler(s) (e.g., employees, managers, supervisors, etc.), generate and effectuate transmission of a notification to the animal's handler to put them on notice of the animal's health condition, and/or provide instruction on next steps for the handler (or other caregiver). For example, the notification may be provided to the handlers in any one or more manners desired—e.g., SMS, email, automated phone call, broadcast, mobile application, etc. —and often via one or more client computing devices 400 associated with the handler.

For example, events component 370 may, obtain a determination from analysis engine 350 and/or treatment selector 360 that cow 702 has developed health condition, Bacterial Infection X, and is in need of antibiotic 4 within the next two hours. Events component 370 may access electronic storage 310 and identify that an employee named Clayton is the assigned handler for the animal, and that Clayton's mobile phone number is (123) 456-7890. Events component 370 may automatically generate a text message using a format such as: “Hi [Handler], [animal ID] you are assigned to appears to have developed [health condition] and is in need of the following treatment: [delivery format/site] of [treatment modality] within the next [time-limit] (i.e., before [local time corresponding to time limit]); [treatment modality] can be found at [location/description]; feel free to call your supervisor [Supervisor] at [number] if you have any questions or concerns.” Thus, drawing on information available to system 1000, events component 370 may generate and facilitate transmission of a text message to Clayton that says: “Hi Clayton, cow 702 you are assigned to appears to have developed Bacterial Infection X and is in need of the following treatment: intravenous delivery of Antibiotic 4 in one of the cow's hind quarter within the next 2 hours (i.e., before 4 pm today); Antibiotic 4 can be found at Barn C in the blue syringes on the south wall, labeled Antibiotic 4 in bold lettering; feel free to call your supervisor Jake at (789) 123-4567 if you have any questions or concerns.”

The above is just one example, as will be appreciated by those of skill in the art, and any variations or modifications to include more or less information may be implemented within the scope of the present disclosure. The same type of message, notification, or alert may be given in any manner, not just in the form of a text message as described in the example above. In some embodiments such messages, notifications, alerts, etc. may be provided via a web portal that a user can access from a desktop computer with an internet connection. In some embodiments such messages, notifications, alerts, etc. may be made available via a mobile app (e.g., AMM App). In some embodiments such messages, notifications, alerts, etc. may be provided in an email, automated voice call, automatic calendar invite, etc. or any other manner desired. System 1000 may be equipped with APIs 380 to interact with any systems or interfaces relevant to such modes of communication, which may in some instances be bi-directional.

As will be apparent to one of ordinary skill in the art upon reading the instant disclosure, system 1000 may be implemented to receive input from a user and propagate updates (based on those inputs) to one or more other elements or sub-elements of system 1000. Thus, system 1000 provides for a dynamic, bi-directional communication system whereby animal monitoring, and tasks related to animal monitoring, may be integrated into a system receiving inputs from various sources (e.g., HMDs, client computing devices, veterinarian input, or other external resources or databases, etc.) and enhancing the efficient and effective care for such animals.

As indicated, events component 370 may operate with enhanced features depending on the resources accessible to it. For instance, system 1000 may include an animal monitoring mobile application (“AMM App”) configured to run on a mobile client computing device 400 (and to communicate with computing platform 330 via an API 380) such as a smartphone. Such an AMM App may provide added mobility and/or enhanced functionality to managers, supervisors, handlers, and other caregivers to improve the efficiency with which care of relevant animals is carried out, and to equip animal caregivers with up-to-date (real-time or near real-time) data about their operation and animals under their care from wherever they are located. Such an AMM App may provide any and all of the tools that may be available to a caregiver via a website (e.g., a dashboard accessible through a web portal), and in some instances may provide additional tools useful to a caregiver when he/she is on-site (e.g., out in the field, on the farm, tending to a given animal, etc.) instead of in the office at a computer.

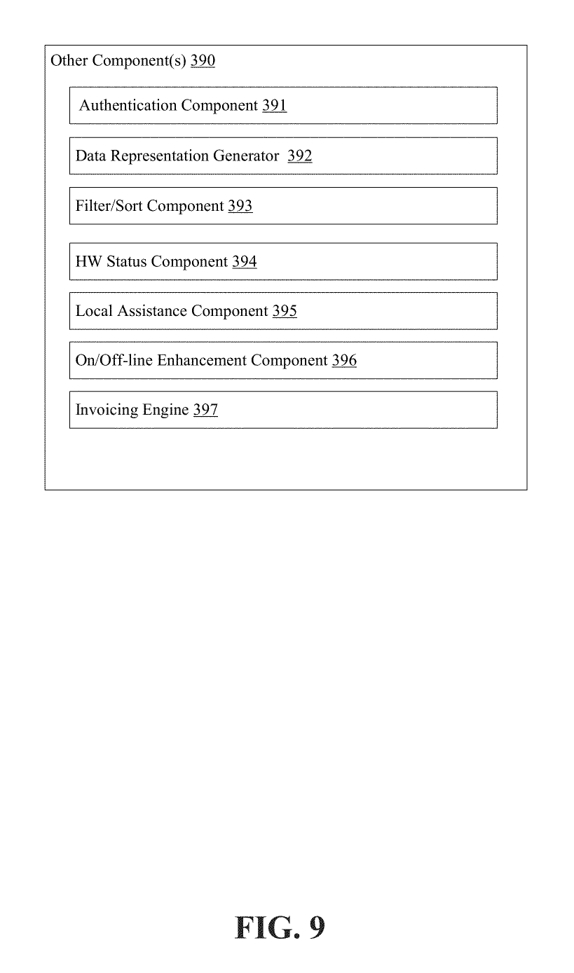

FIG. 9 is a block diagram various examples of other components 390 that may be employed in one or more embodiments of the present disclosure. Just as the components shown in FIG. 8, the components in FIG. 9 may be executed on computing platform 300, executed on client computing device 400, executed on other computing resources within system 1000, or any combination of the foregoing (e.g., in any distributed, parallel, or duplicative computing model). As shown, other components 390 may include one or more of an authentication component 391, list generator 392, filter/sort component 393, hardware (HW) status component 394, local assistance component 395, on/off-line enhancement component 396, and an invoicing engine 397.

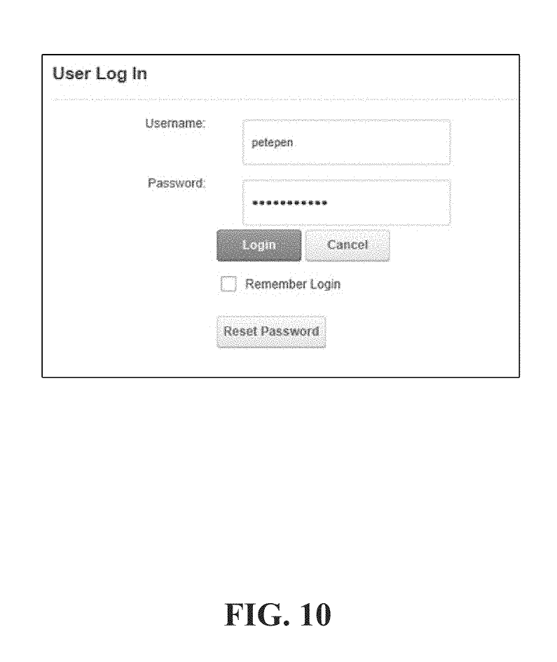

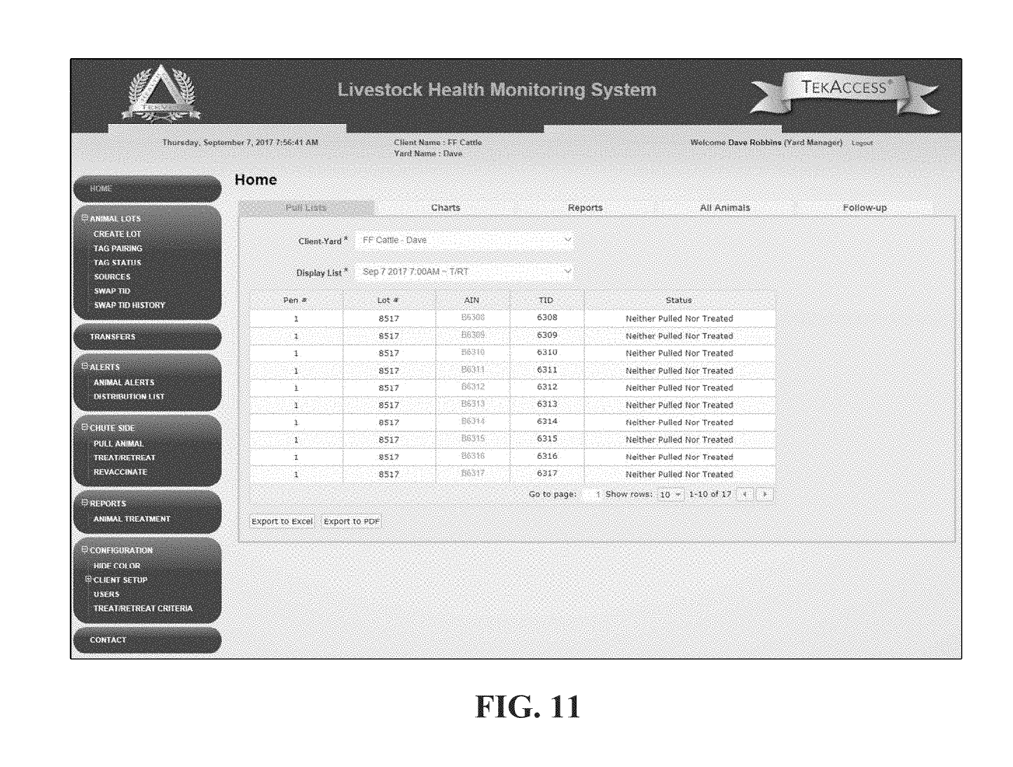

Authentication component 391 obtains credentials inputted by an end user (e.g., an animal caregiver registered with system 1000 (i.e., one who has created an account to obtain access to system 1000)) via a client computing device 400, determines if the credentials entered satisfy authentication criteria. Such credentials may be obtained via acquiring the input entered into editable fields provided by a GUI presented to the user on a client computing device 400, for example, such as shown in FIG. 10. Upon determining that the credentials entered satisfy authentication criteria, the authentication component may allow the user to enter through a portal (e.g., login via a web portal on a website, login to an AMM App on a smartphone) to view information relevant to that user in system 1000, the portal providing access to an interface (e.g., a GUI) including one or more of static, dynamic and active content conveying information from system 1000 relevant to the particular user. An example of such a GUI, which may be provided via a secure web domain, is shown in FIG. 11. Such a GUI may sometimes be referred to herein as the a dashboard. The dashboard may be customized for a particular user based on the user's role in the operation, and a data representation generator 392 component may tailor what is provided to the given user accordingly. As shown in FIG. 11, the dashboard may include tabs that give the user acces to Pull Lists, Charts, Reports, Animals, and Follow-up items (follow up notes/task items). These will be referred to in more detail below.

Data Representation Generator 392 determines which registered user is associated with the credentials inputted via a client computing device 400, obtains information accessible to system 1000 relevant to the registered user (e.g., treatment modality information for one or more animal's under the given user's care), and optionally generates one or more display objects or other representations of the relevant information. The data representation may take any form, including a graphic, a plot, a table, a list, an audible sound, a video, a picture, a photo, a text notification or alert bubble, or any other representation or indication of at least a portion of the information accessible to system 1000. In some embodiments the data representation is given a particular name or otherwise associated with a particular tab, button, or menu item (e.g., on the user's dashboard) that a user may select to view and interact with the data underlying the representation.

For instance, the data representation generator 392 may recognize that the user that just logged into the system 1000 is a handler in charge of six thousand pigs distributed among six lots (lots 1-6), each lot including a single pig pen (pens 1-6). Data representation generator 392 may provide a menu of various data representations relevant to the particular user. Such menu items may be predefined by the user (e.g., the handler), a user's supervisor, or provided as a default that may be later customized. A menu may include buttons or tabs associated with different data representations. For example, an example menu may include buttons associated different list or table styled data representations relevant to the particular user—e.g., a Pull List, Non-Conforming List, a Consecutive Miss List, and the like.

A Pull List may include a listing of animals that need to be pulled from their respective pens to receive a treatment (or retreatment as the case may be). As described above, the treatment selector 360 may have determined the treatment to be provided to the animal to be pulled on a particular date, which may in turn have been determined in response to analysis engine 350 evaluating one or more physical parameters about the animal via acquisition component 340. The end user may select a menu item associated with his/her Pull List and be provided with the data representation generated by data representation generator 392 (e.g., tap the screen of his/her mobile device on an AMM App menu item titled, “Today's Pull List” for example, and be taken to a page within the AMM App that provides a Pull List of animals that that particular end user should pull from their pens that day for treatment; or click on a tab and/or dropdown menu item through the dashboard accessed through a website viewed on an internet browser; or other access method).

Such an example Pull List is shown in FIG. 11. As shown, the list may include active content, such as a clickable box region that allows a user to click the box associated with a given animal to deem the animal pulled for treatment. That way, as the end user tours respective lots pulling the animals on his/her Pull List, he/she can mark off the animals from the list as they go. If a user doesn't get all the way through their list in a given day, one or more of the leftover animals that should've been pulled may either be added to the next day's pull list or be added to a different list (that may be termed, for example, a Nonconforming List or Consecutive Miss List, depending on criteria for each) or both. Data representation generator 392 may provide additional information about animals in conjunction with such lists or other graphics (e.g., Animal AIN, TID, Pen Number, Lot Number, Yard, etc.)

As noted, Nonconforming List may include a listing of animals that should have been pulled on a previous day but for one reason or another were not actually pulled (e.g., the ranch handler ran out of time in the day, the prescribed medication was out of supply, etc.). The end user may select a menu item associated with his/her Non-Conforming List and be provided with the data representation generated by data representation generator 392 (e.g., tap the screen of his/her mobile device on an AMM App menu item titled, “Today's Non-Conforming List” for example, and be taken to a page within the AMM App that provides a Non-conforming List of animals that the particular end user should prioritize as he/she proceeds to pull animals from their respective pens for treatment or retreatment; or click on a tab and/or dropdown menu item through the dashboard accessed through a website viewed on an internet browser to access such list; or other access method).

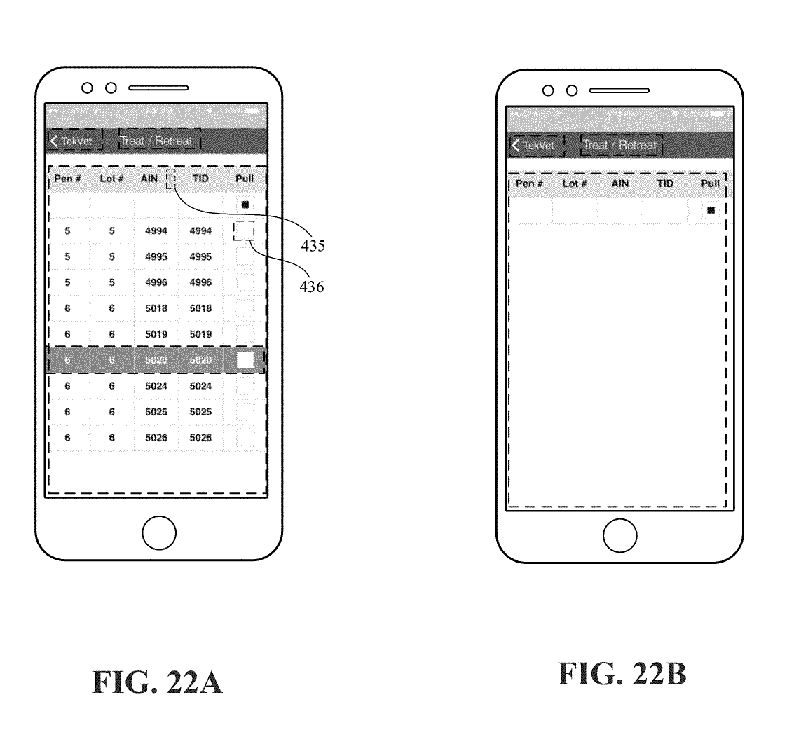

Filter/ort component 393 may filter and organizeinformation in conjunction with data representation generator 392 in accordance with a user category, a preference or a selection. For example, as shown in FIG. 22A, an AMM App running on a client computing device 400 may include a sorting button 435 that, when selected by a user, rearranges the rows in ascending numerical order of each animal's animal identification number (AIN) (i.e., the number assigned to the animal by the operator). The sorting button 435 may, if selected again, may rearrange the rows in descending numerical order of each animal's AIN. Of course, although sorting button 435 is shown in FIG. 22A associated with the AIN column, filter/sort component 393 may organize the information provided by the data by applying any sorting rule to any column, row, or other feature of the data represented. Although the sorting rule applied and illustrated in FIG. 22A is simply a numeric ordering based on the AID number, any rule may be applied such that the data is represented in the manner desired. For instance, the sorting rule may provide for the list to be ordered based on temporal imminence of treatment (e.g., ordering animals needing treatment within the next hour before animals needing treatment anytime in the next six hours, for instance).

In another example, the filter/sort component 393 may apply a sorting rule that provides for the list to be ordered based on financial considerations (e.g., ordering the Pull List such that animals of greater financial value to the operation appear before those that are of less financial value). In another example, the sorting rule may provide for the list to be ordered based on how contagious the health condition is that the analysis engine identified for a given animal (e.g., ordering the Pull List such that animals diagnosed with a highly contagious disease are placed before animals diagnosed with non-contagious or less contagious diseases). The foregoing are just examples that are not intended to be limiting. Any other sorting rule may be employed to effectuate a desired sorting of data provided by the data representation generator 392 in conjunction with the filter/sort component 393.

Moreover, in some embodiments filter/sort component 393 may be adapted to filter what information is provided or otherwise presented to an end user on their client computing device 400 (e.g., by eliminating information from view based on a filter rule, showing only a certain type of information as desired based on a filter rule, compiling or preparing one or more lists or other graphics based on a filter rule, etc.). That is, filter/sort component 393 may apply one or more filter rules to aid the user's analysis of the information presented, or to aid the user in evaluating a diagnosis for a given animal.

It should be noted that system 1000 may aggregate information about an animal and—in conjunction with one or more of analysis engine 350, events component 370, other components 390, or any other elements of system 1000—apply one or more rules (e.g., aggregation rules, filter rules, sorting rules, etc.) to generate one or more data representations about aggregated data across a herd, yard, lot, pen, the operation generally, or other designated entity of an operation. Such data representations may be based on an aggregate view of the information for the given designated entity.

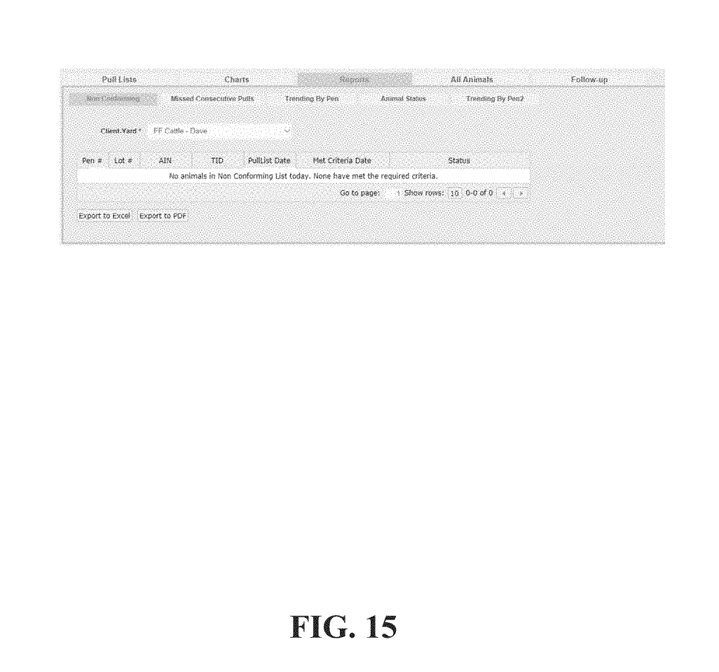

For example, as shown in FIG. 15, one or more embodiments of the present disclosure may include a dashboard where a user can select what health conditions are trending by pen. That is, one or more components of system 1000 may aggregate information across all the animals in given pen, and make an assessment based on the aggregate information. For instance, the aggregate information may include health conditions developed in 1000 animals in pen 5 over the last week. The information for Monday may indicate that one animal in pen 5 developed health condition A. The information for Tuesday may show that 6 animals in pen 5 developed health condition A. The information for Wednesday may show that 40 animals in pen 5 developed health condition A. Thus, system 1000 may aggregate the aforementioned information and, based on the exponential increase in onset of health condition A in pen 5, make an assessment that the onset of health condition A in pen 5 shows an increasing trend (e.g., is trending upward). Accordingly, if an operator were to click on “Trending by Pen” tab in the dashboard in FIG. 15, for example, the GUI may present such operator with a chart of the trend for health condition A among pen 5, and a notification and/or recommendation to the user such as “Health Condition A appears to be spreading quickly throughout your herds in Pen 5, you might consider separating animals with [AIN numbers X-Y] from animals with [AIN numbers W-Z]” for example.

Such aggregation of data may be aggregated over any period of time for any number or group of animals overseen in a given operation. An operator may drill down further into the information as made clear from the figures. For instance, FIG. 11 shows that system 1000 is maintaining data for “1-10 of 17”—the number 17 in this example shows the user exactly how many animals are sick in the yard, for example, and system 1000 may keep track of that totals on one or more lists generated over time. In other words, the data collected by system 1000 may be stored and aggregated over time to identify trends and/or make predictions to aid an operator in understanding or receiving an overall high-level view of their yard and animals. Such data aggregation, evaluation, and analysis may include any one or more predefined operations including data summarization, calculation, multi-dimensional analysis, statistical analysis, trend analysis, and other algorithms.

Accordingly, system 1000 may provide managers or other operators with high-level information (e.g., broader assessments such as health trends, general status about a yard, lot, pen, herd or other designated entity) that the operator may drill down into (e.g., via selection) to obtain lower-level (e.g., more specific information). An operator may request this information by clicking a button on their GUI, or in some embodiments via voice command (e.g., speaking a request into their client computing device 400 that system 1000 is configured to recognize and respond to, such as, “how is my yard doing this week,” or “what are the health trends in the pens I'm in charge of,” for example).

In some embodiments, the dashboard accessed via client computing devices 400 may provide an input field through a graphical user interface provided to a user, obtain input from a user via the input field, and update or otherwise synchronize the information throughout the system 1000 in real-time, near real-time, periodically on an update cycle, or whenever the client computing device 400 is connected to the client computing platform 300 over a network or communications link. In some embodiments, an input field may be displayed as part of the data representation provided by data representation generator 392. An input field may be configured to receive text input (such as the login input field shown in FIG. 10), a selection input (such as the checkbox field 436 in FIG. 22A), or any other input. In some embodiments, the dashboard may be configured to receive image, video or audio data from a peripheral device that is either operatively coupled to the client computing device 400, or part of the client computing device 400 (e.g., a camera or speaker module of the client computing device).

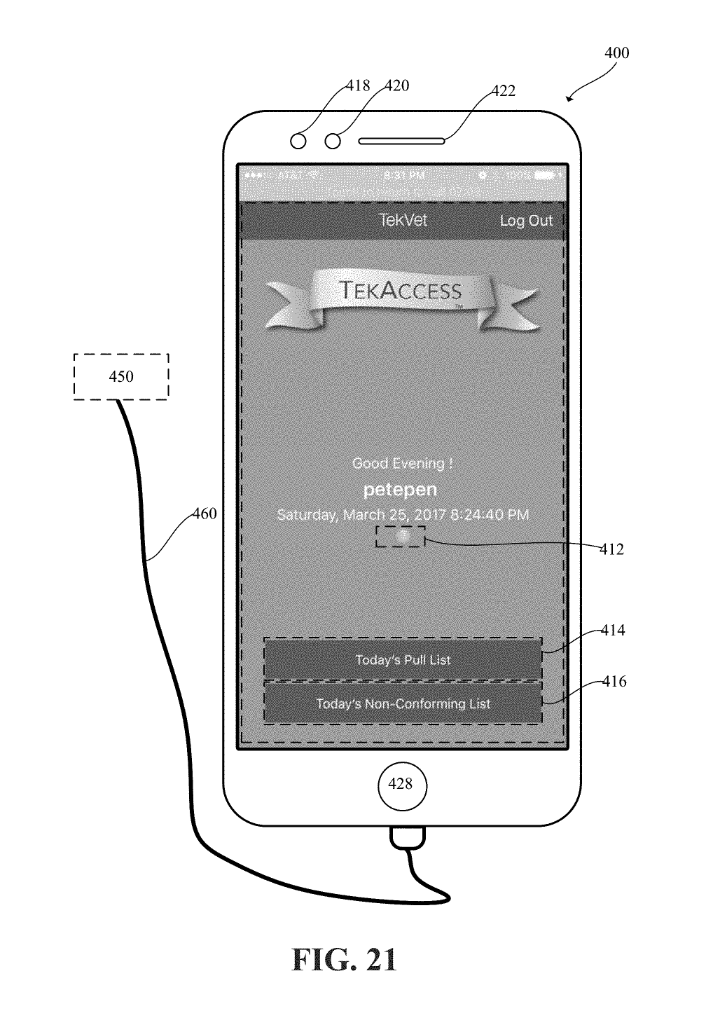

In some embodiments, the dashboard and the client computing device are adapted to interface with a peripheral device that may be connected over a wired or wireless interface with client computing device 400, as shown in FIG. 21 by symbolic peripheral 450 connected to client computing device 400 over cable 460. Thus, a peripheral device may be coupled with a client computing device 400, the peripheral device may be adapted to obtain in-field information about an animal. For example, in some embodiments a peripheral device may include an electronic medical device that requires an end user to maneuver it into the proper position with respect to the animal (e.g., in an animal's mouth for a temperature measurement, or saliva acidity measurement, etc.) so that a proper reading may be taken and provided to the system 1000 via client computing device 400.

Referring back to FIG. 9, computing platform 300 may include a HW Status Component 394. HW status component 394 may obtain hardware status information about an HMD 100 and optionally provide an alert, a notification, or other indication of such status to a user via user's client computing device 400 (e.g., via a web portal or dashboard, an AMM App portal or dashboard, etc.). For example, if system 1000 determines that an HMD 100 is getting low on battery power and needs a replacement, HW Status Component 394 may obtain such information and make it accessible to a user via the user's client computing device 400. In some embodiments, the low battery status may be indicated in an alert (e.g., via text message, voicemail, calendar invite), or a notification (e.g., a notification within AMM App or web dashboard, for example, or adding the HMD ID information to an HDM Repair List accessible to user), or some other indication (e.g., changing the pixel color at a predefined zone of the display near a battery icon on the display of a client computing device 400, such as changing the color from green to red, red indicating the HMD is in need of battery repair, replacement, or recharge, and green indicating the battery is operating properly, for example). In some embodiments, a battery icon depicting the remaining amount of battery power left, may be provided within a data representation generated by data representation generator 392.

Local Assistance Component 395 obtains information about the location of other employees on the operating lot that may be summoned (e.g., by text, call, alert, notification, or otherwise) to handle a particular task or to assist another user in handling a particular task. The ability for one user to summon the help of another user may, in some embodiments, be limited by their user category. In other embodiments, any employee or caregiver of an operation can send a request for assistance to any other employee or caregiver of the operation. In some embodiments the user may select among one or more employees available for summoning for assistance. For instance, a caregiver named Handler 1 may log into the AMM App on their phone (their client computing device 400), click on the button for the pull list for the day, and begin to proceed throughout respective lots to pull the identified animals from respective pens for treatment and mark them off the list (e.g., via input fields 436 shown in FIG. 22A, for example). After pulling a given animal for treatment, Handler 1 may struggle to deliver the particular treatment modality (e.g., a drug to be delivered intravenously) and need another person to help hold the animal in place while the treatment modality is delivered. Handler 1 may open his AMM App, for example, and select a mapping button that opens a map (e.g., an aerial map) with one or more pins showing the location of other caregivers within a certain distance, e.g. within 3000 feet of Handler 1, or otherwise within the vicinity of Handler 1. For instance, the map may include three pins, one for Handler 3, one for Handler 4 and one for Handler 7. Handler 1 may tap on or otherwise select the pin associated with one of the other Handlers within the vicinity, which may effectuate an automatic notification or alert (e.g., a help request) to the client computing device 400 of the Handler associated with the pin Handler 1 selected, for example, to Handler 7's client computing device 400. In some embodiments the summoned caregiver, in this example Handler 7, may be given an option, through their AMM App or other GUI communicatively coupled to system 1000, to accept or reject the request. Notifications indicating acceptance of a summons, rejection of a summons, an estimated time of arrival of the summoned handler, reassignment/delegation of a summons request, or any other indication or communication useful for the given application. In some embodiments an AMM App may open a two way communication channel (e.g., initiate a text conversation, a video chat, or otherwise) between the caregiver who made the summons request, and the caregiver responding to the request.

Though these other components 390 may be executed on computing platform 300, in some embodiments all or some of these may be implemented (e.g., executed), in whole or in part, at the client computing device 400.

In some embodiments, other components 390 may include an on/off-line enhancement component 389. On/Off-line enhancement component 389 may detect when a client computing device 400 is within, or proceeding into, an area with low connectivity (e.g., poor signal strength on a cellular network, for example), and automatically initiate a synchronization and/or download of relevant data from computing platform 300 to a local storage sector on client computing device 400. Thus, when a user proceeds into an area of low or no connectivity, they can still access recent information available through system 1000. In some embodiments, On/Off-line enhancement component 389 may detect that a user's route to a known destination will end in an area with no connectivity, and trigger synchronization and/or download before the client computing device 400 enters such area. In other embodiments On/Off-line enhancement 389 component may be location agnostic, and periodically run a synchronization and/or download operation to store relevant information from system 1000 on a regular basis (e.g., every 5 hours, every 24 hours, etc.).

In other embodiments On/Off-line enhancement 389 component may perform a synchronization and download operation in response to an occurrence of any one or more system events, and update, upload, or change in any one or more elements of system 1000, or any other predefined occurrence or criteria. In some embodiments, On/Off-line enhancement component may perform a synchronization and/or download operation in response to input provided by a user. For example, if a user knows they are about to enter an area with low connectivity, they may press a button (e.g., on the GUI of the AMM App) or otherwise make a selection to initiate synchronization and/or download.

In some embodiments On/Off-line enhancement 389 may operate automatically (e.g., initiate updates, synchronization, and/or download in the background and continuously propagate updates through system 1000). For example, system 1000 may automatically synchronize and/or propagate information throughout one or more other elements of system 1000 in real-time, near real-time, or periodically based on a combination of factors (e.g., such as system configuration, frequency of underlying data changes, system load, network availability, network congestion, time of day, user preference, network connection upload and/or download speeds, and the like). In other embodiments, as noted, it may operate at the request of a user (e.g., manually refreshing data upon user request.)

In some embodiments on/off-line enhancement component 389 of system 1000 may include a synchronization check feature that applies a conflict resolution procedure. Consider an example where multiple users perform actions relevant to the same or related data while in an offline-mode of operation, then later system 1000 attempts an update when one or more of the multiple user devices come back online, but an update from one device conflicts with an update from another device for the same or related data. To handle this situation, system 1000 may employ a conflict resolution procedure. Such conflict resolution procedure may include one or more of: providing a report of the conflict to an operator (such as a manager of two or more handlers who created the conflict in the update), permitting the operator to select which update to adopt, and propagating the operator selected update throughout the system. In some embodiments the conflict resolution procedure may be automated for certain conflicts. For instance, system 1000 may determine that the conflict was a result of a first handler entering information in error (e.g., a handler not overseeing an animal with AIN 5645 entering an update for AIN 5645 when such handler was not even located on the lot where animal with AIN 5645 was located), and thus adopt the update from a second handler. Operators may provide any conflict resolution criteria to enhance the workflow of system 1000 and avoid conflicts when utilizing on/off-line component 389 features. The conflict resolution criteria may be based on one or more of: seniority or role of caregivers providing the update, timing of the update, location information associated with the update, animal data associated with the update, the history of errors made in updates provided by a particular caregiver, or any other criteria pertinent to the given operation.

Invoicing engine 397 determines if and/or when a payment triggering event has occurred, and generate or provide one or more of an invoice (or a line item for an invoice), a bill (or a line item for a bill), a payment, or other financial/transactional record or operation associated with the payment triggering events. In some embodiments, a management entity may provide implementations of system 1000 to a plurality of operations (who may be clients of the management entity, for example), and may expect payment from the individual operations in exchange for use of system 1000 or any one or more elements or sub-elements of system 1000. A management entity may comprise one or more computing resources operatively coupled with (and in some embodiments considered part of, or an extension of, system 1000). Such management entities may establish payment triggering events (e.g., stored as payment triggering event rules in a memory or electronic storage of system 1000, for example) that, upon occurrence, may be detected by invoice engine 397. Invoice engine 397 may then generate one or more of an invoice (or a line item for an invoice), a bill (or a line item for a bill), a payment, or other financial/transactional record or operation associated with the payment triggering event.

For example, a management entity may desire to charge operators a fee each time a new HMD 100 is deployed on the operators operation. In such an example, a payment triggering event rule may be satisfied whenever system 1000 detects that a new HMD 100 has been paired with an animal. Such a determination could be made by detecting when an HMD 100 with a new electronic ID comes online (e.g., within a given operation) or is newly paired with an animal, and generating an electronic line item on an electronic version of an invoice, bill, transaction record, or other representation of the event and corresponding payment for the operator.

The payment triggering event rule may be based on any triggering event conditions the management entity is interested in, such as the passage of time (e.g., monthly billing), an order of materials (e.g., order of additional receivers), performance of services (e.g., processing a medication shipment), or usage of physical devices (e.g., the amount of time HMD 100's are used in a given time period, the number of HMD 100 transmissions that occurred at a given lot/pen/herd, etc.), or any other rule or criteria desired by a management entity or operator.

System 1000 may provide an electronic summary of charges and payments due via a tab available to the operator in the operator's dashboard (e.g., accessible via an AMM App, or a web portal, etc.). Invoicing Engine 397 may generate a summary of charges and provide the same to an operator, e.g, via the operator's dashboard accessible through their client computing device 400, via a notification delivered through the operator's AMM App on their client computing device 400, etc. Invoicing Engine 397 may generate or provide such invoices, bills, transaction records, or other representations of the events and corresponding payments as the events occur, or on regularly scheduled intervals (weekly, monthly, quarterly, annually, etc.) including a summary for that interval.

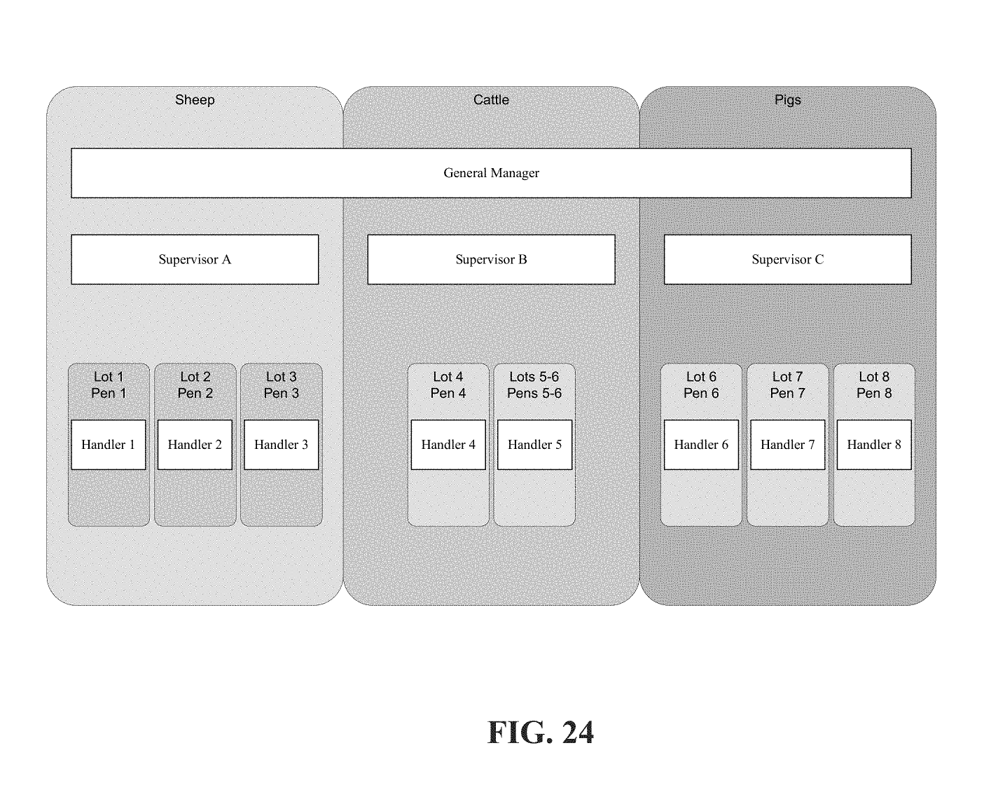

As noted above, in some embodiments, system 1000 may provide access to information via an animal management application (AMM App). In some embodiments AMM App may run on a server that is part of computing platform 300, and be available to client computing device 400 over the web. In some embodiments, AMM App may be downloadable to a client computing device 400 and may run atop an operating system of the client computing device 400. Operators, caregivers, or other end users may register or otherwise create an account with system 1000 such that system 1000 recognizes them when they log-in (e.g., via AMM App, via a web portal, etc.). AHM App may be adapted to provide relevant information to an end user. In some instances, the information provided to the end user is tailored or limited depending on who the user is or what the user's predefined role is in relation to system 1000. In some embodiments an end user is registered within a particular category of user, and the information provided to or otherwise available to the end user is tailored or limited depending on the users category. An example of various user categories that may be employed in one or more implementation are shown in FIG. 24 (user categories including, in this example, General Managers, Supervisors, and Handlers).

Referring to FIG. 24, for example, suppose a farm operation includes a herd of four thousand sheep, a herd of three thousand cattle, and a herd of five hundred pigs—each animal equipped with an HMD 100. Suppose further that the ranch operation employs a general manager to oversee the entire ranching operation; three supervisors—supervisor A to oversee the sheep, supervisor B to oversee the cattle, and supervisor C to oversee the pigs; and nine animal handlers—handlers 1-3 reporting to supervisor A, handlers 4-5 reporting to supervisor B, and handlers 6-8 reporting to supervisor C. Handler 1 may be responsible for sheep on Lot 1 in Pen 1, handler 2 may be responsible for sheep on Lot 2 in Pen 2, and handler 3 may be responsible for sheep on Lot 3 in Pen 3. Handler 4 may be responsible for cows on Lot 4 in Pen 4, handler 5 may be responsible for cows on Lot 5 in Pen 5 and on Lot 6 in Pen 6. Handler 6 may be responsible for pigs on Lot 6 in Pen 6, handler 7 may be responsible for pigs on Lot 7 in Pen 7, and handler 8 may be responsible for pigs on Lot 8 in Pen 8.

Suppose Handler 5 logs in to system 1000 via an HMM App on his client computing device 400 and selects a Pull List menu item to view a list of animals in his Pen that need to be pulled for the day for treatment. Handler 5 may see a data representation similar to the one shown in FIG. 22A. Each row may be associated with an animal that needs to be pulled for the day and treated. As handler 5 proceeds to tour his lot pulling the listed animals from their respective pens, he may select checkbox 436 fields associated with animals pulled and they may then vanish from the listing. Once handler 5 pulls all the animals in the Pull List and marks their respective checkbox fields indicating the animal has been pulled, the user may see the data representation in FIG. 22B indicating there are no more animals to be pulled by that caregiver.

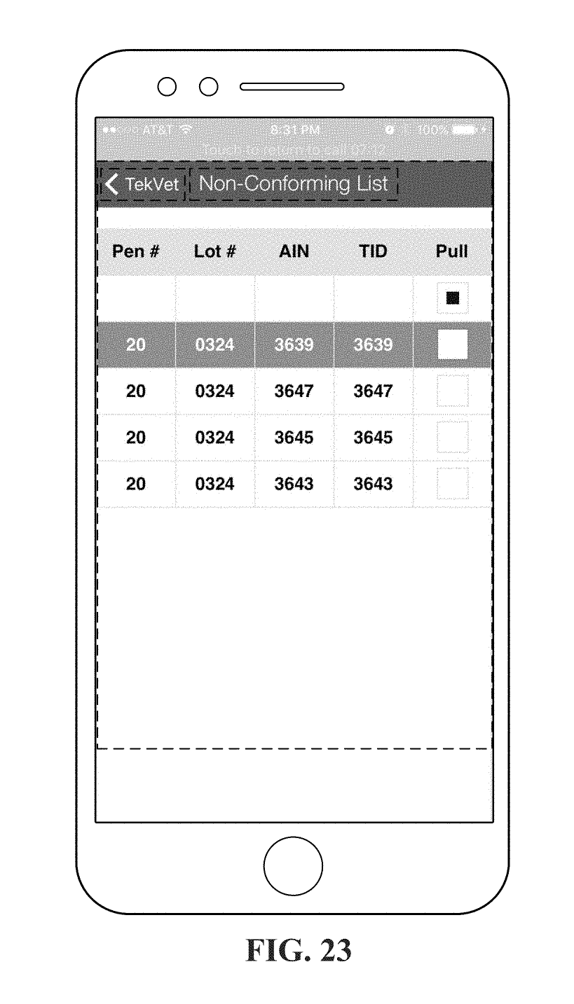

FIG. 23 illustrates an example Non-Conforming List in accordance with one or more embodiments of the present disclosure.

As explained herein, in accordance with one or more embodiments of the present disclosure an AMM App, web dashboard, or other GUI accessible to a mobile client computing device 400 is provided for enhanced notification and communication features of system 1000. For example, if a notification is provided to a user's client computing device 400 via an AMM App, for example, the AMM App may include options for the handler to (i) obtain on demand delivery information describing or depicting how to deliver the treatment modality to the animal (e.g., written instructions, linked video tutorial, etc.), (ii) obtain on demand location information depicting or describing where the animal is on the property in real time, or near real time (may include map pins and route information based on GPS, trilateration, or any other location derivation process, any one or more of which may utilize or be based upon information about (e.g., geo-location of a receiver) or from (e.g., information received by a receiver) receivers 200, (iii) activate, automatically or on demand, one or more visible light sources or audible audio sources associated with the animal (e.g., the animal's HMD 100) to assist the handler in locating the animal (iv) activate, automatically or on demand, one or more visible light sources, audible audio sources, and/or haptic feedback sources (e.g., vibration motors) at a client computing device (e.g., the a handler's smartphone) to assist the handler in locating the animal (e.g., a speaker of the user's smartphone providing an audible indication such as “the animal is within 2 meters of your present location,” or “the animal is to your left by 5 yards,” for example, or a vibration motor of the user's smartphone causing vibration when the user is close to the animal (e.g., within X meters, or some other predefined proximity threshold), or a vibration motor of the user's smartphone causing vibrations of the handler's smartphone with increasing intensity as the user gets increasingly close to the relevant animal), (v) obtain warning information about the animal such as the animal's level of aggressiveness, (vi) reassign one or more tasks to another backup handler if for one reason or another the first handler cannot complete the required tasks, (vii) request assistance from nearby handlers on the property during a task, (viii) scan the barcode on the relevant treatment modality used (the Antibiotic packaging) when pulled from the stock room so that stock information can be updated, (ix) confirm when any one or more relevant tasks has been completed (and optionally to stop reminder alerts if reminder alerts had been set), (x) accept a calendar invitation that places the work item in the users mobile calendar with an associated alert/reminder, (xi) provide customized comments or notes about the animal (e.g., “the animal's ear appears to be cut and bleeding, so the temperature readings from the sensor may have been erroneous,” “when I showed up to deliver the antibiotic, the animal was dead,” or “the cow appears to have a cut on its left leg and walks with a considerable limp that a vet should probably treat, I've uploaded a picture attached here” (e.g., picture accessible via hyperlink on “here” term)), or (xii) any other desired interactivity or feature, including but not limited to any and all interactivity and features described herein.

An AMM App may be configured with a user interface that enables an end user to receive and interact with information from system 1000 while moving about in the field (e.g., on the ranch). Thus, additionally or alternatively to providing end users with a web portal (e.g., a website) through which to access relevant information about a given animal, herd, or operation generally, system 1000 also contemplates the inclusion of a mobile application (e.g. AMM App) that allows a user to easily access and interact with the information of system 1000 while moving about in the field. The AMM App may have a graphical user interface including one or more of static content, active content and dynamic content—each of which are commonly understood by one of ordinary skill in the art. Computing platform(s) 300 may include a server running an Application Programming Interface (API), the API operating to enable communication between the AMM App running on the client computing device 400 (e.g., a mobile phone, a tablet, etc.) and the computing platform(s) 300, including but not limited to the business logic relevant to the computing platform(s) 300, or any servers or databases that comprise the computing platform(s) 300. FIGS. 22-24 illustrates an exmaple GUI illustrating some of the features discussed herein with respect to an example AMM App or other GUI or other program running atop a client computing device 400 operating system.

AMM App may utilize the computing resources of the client computing device 400 it is running upon to execute functionality that might in other instances be executed by computing platform(s) 300. Indeed, any one or more of the acquisition component 340, analysis engine 350, treatment selector 360, events component 370, or other components 390 may be executed by a processor local to the client computing device 400 rather than the computing platform(s) 300. Alternatively, in some embodiments execution of the one or more of the machine-readable instructions relevant to any one or more of the foregoing may be run on either or both of the computing platform(s) 300 and one or more client computing device(s) 400. That is, system 1000 may employ a distributed computing scheme to efficiently utilize the computing resources available to it.

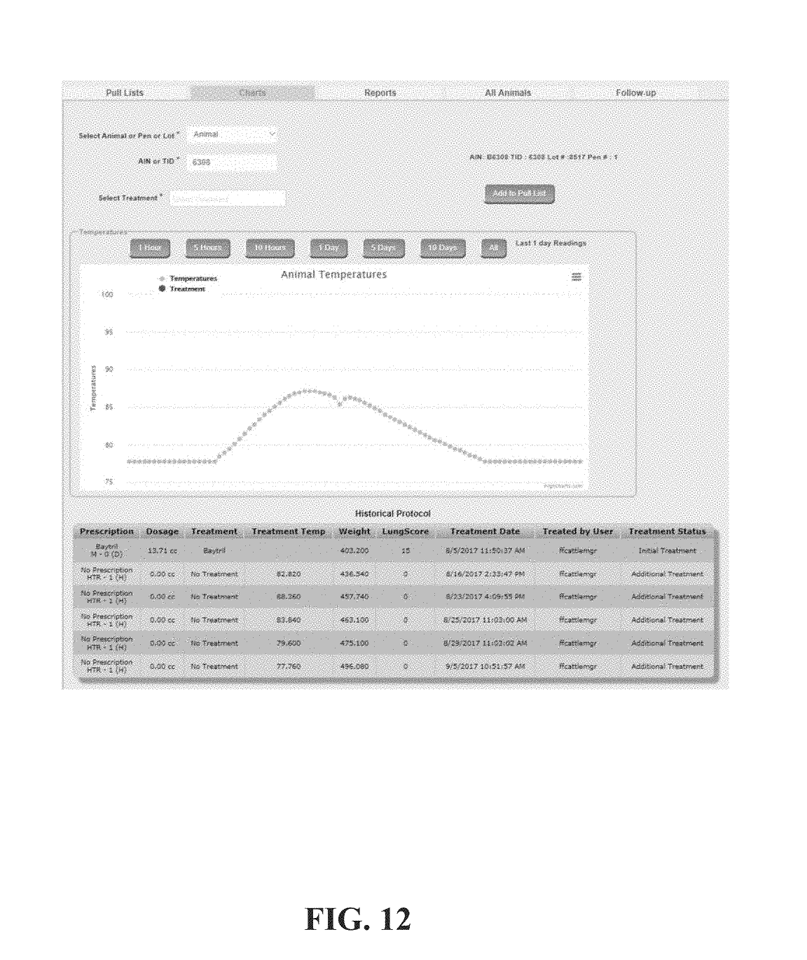

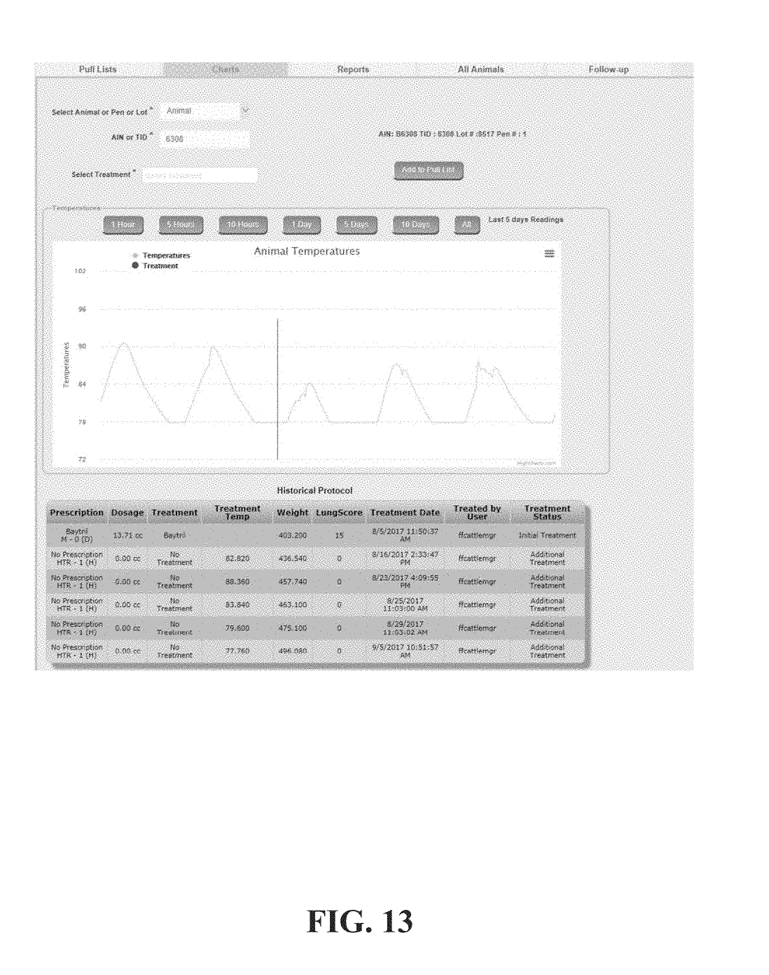

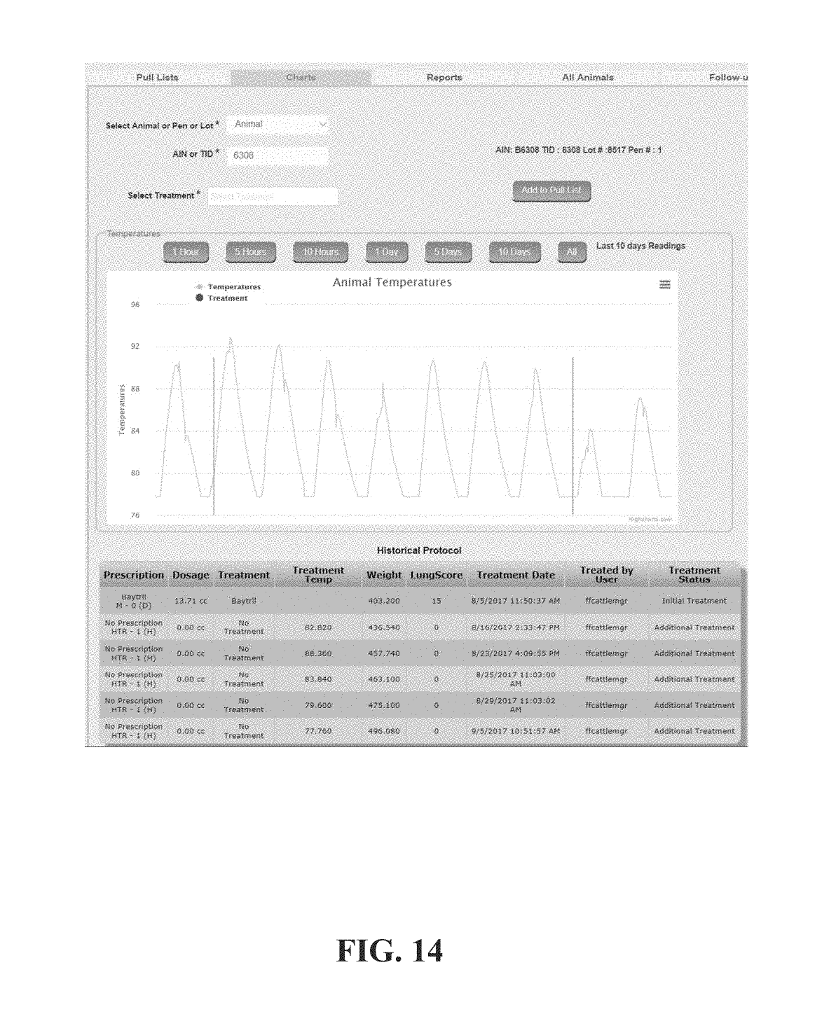

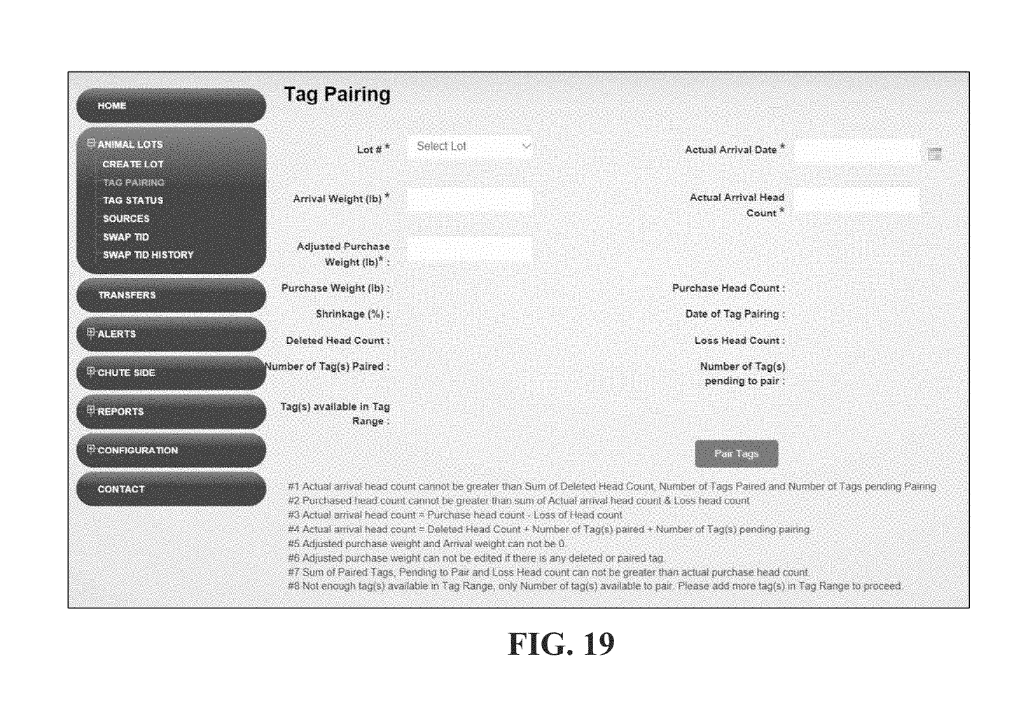

Similarly, any and all of the functionality described herein may be available through a web portal that provides a user with a dashboard, as noted previously, providing access to information available through system 1000 that is relevant to their role in a given operation. Example screens implementing some embodiments of how the technology disclosed herein may be provided are included in FIGS. 11-20. FIG. 11 illustrates an example dashboard GUI having various tabs and menu items allowing a user to navigate through, consume, use and interact with information available through system 1000. FIG. 11 shows an example Pull List. FIG. 12 illustrates an example chart that data representation generator 392 may generate for a given animal depending on the user's selections. Here data representation generator 392 has created a graphic that shows a visual representation of an animal's (animal associated with AIN 6308) temperature readings over the last day, as well as a table of other information related to the animals health (e.g., prescriptions, weight, lung score, treatment dates, etc.). FIGS. 13-14 show a similar example chart that data representation generator 392 may generate for a given animal, here showing temperature readings over the last 5 days and last 10 days respectively. FIGS. 13-14 also show a vertical line delineating a point in the timeline when the animal was treated. Thus, a user may consume historical information as desired.

As noted previously, historical information or any other information about given animals may be stored securely within system 1000 (e.g., utilizing digital signatures or cryptographic protection) to prevent information in animal records from becoming compromised, or reduce the likelihood of the same. In some embodiments data security measures preventing unauthorized access or modification to data stored upon or accessible to system 1000 may include procedural or policy lockouts (i.e., user permissions or database safeguards, including permissions based on user authentication by authentication component 391). Some embodiments may include multi-factor authentication procedures (e.g., two factor authentication) to enhance security of animal data (e.g., animal records 312). In some embodiments a third party may desire to authenticate or otherwise validate the accuracy, origin, or secure character of one or more pieces of animal information (or any other information available to or stored upon system 1000, e.g., animal records 312) such that the information may be represented with verifiable integrity (as an electronic medical record) to, for example, the FDA or other government agency, courts, consumers. System 1000 may implement any security protocols, digital signatures or certificates, authentication, permissions, or protections, including those presently known in the art and/or any later arising, to establish the authenticity, security, and accuracy of any one or more pieces of information associated with animals monitored by a system 1000.

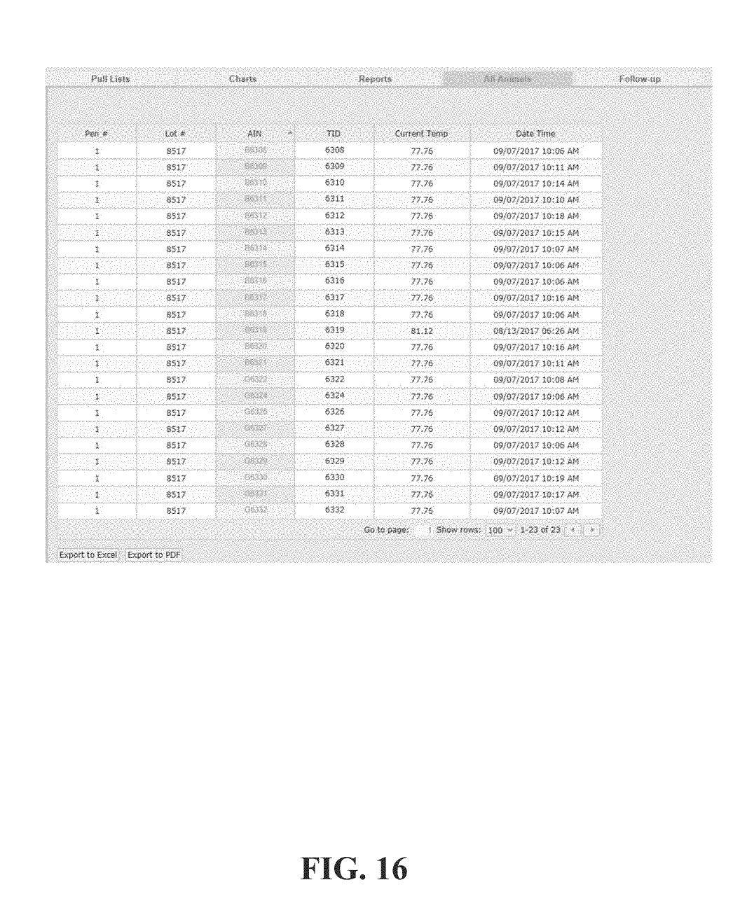

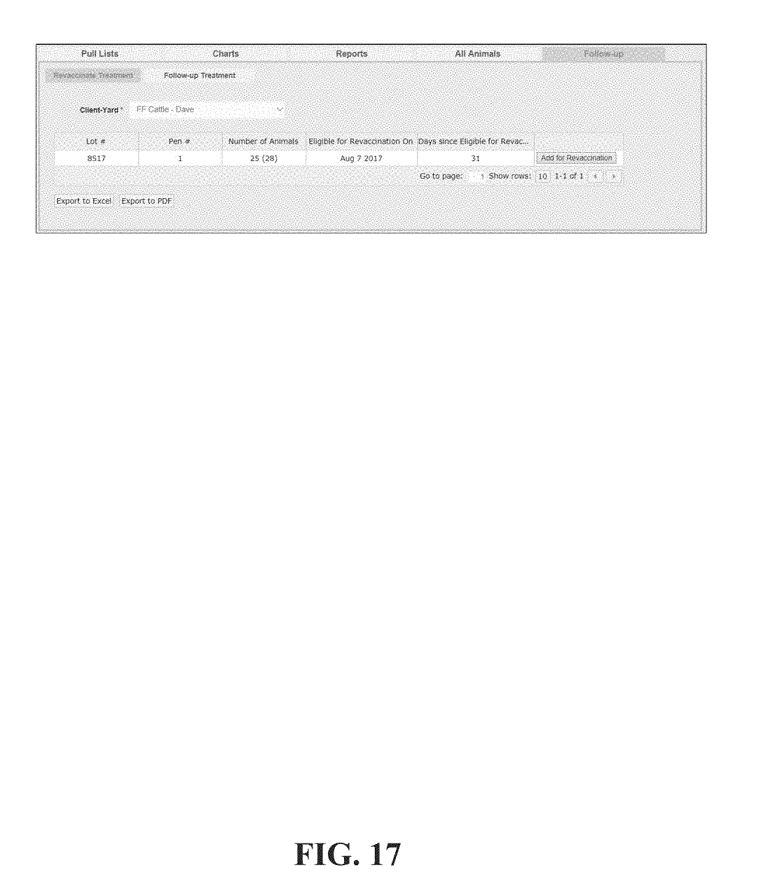

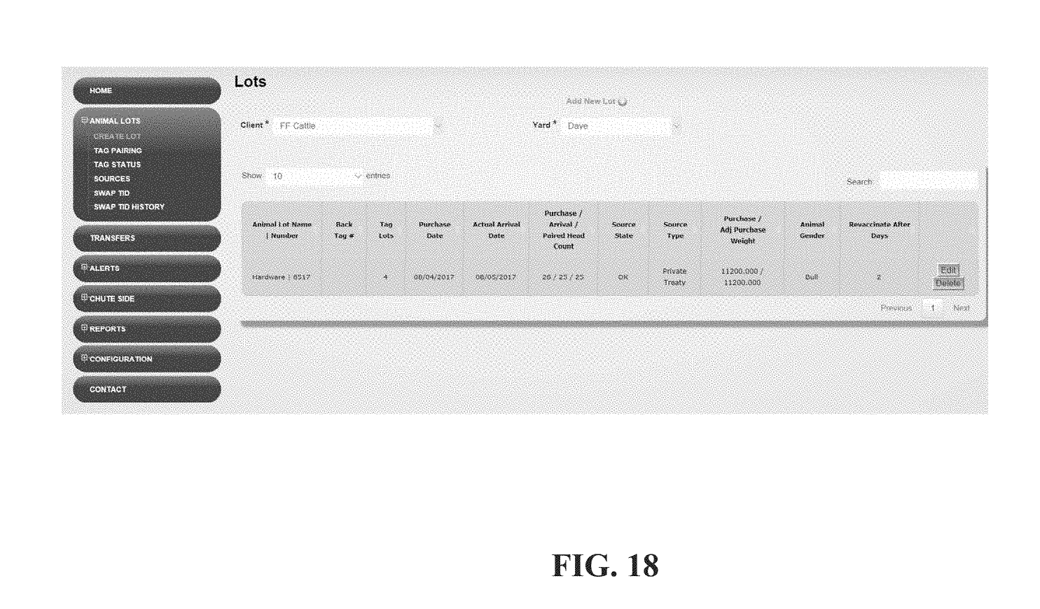

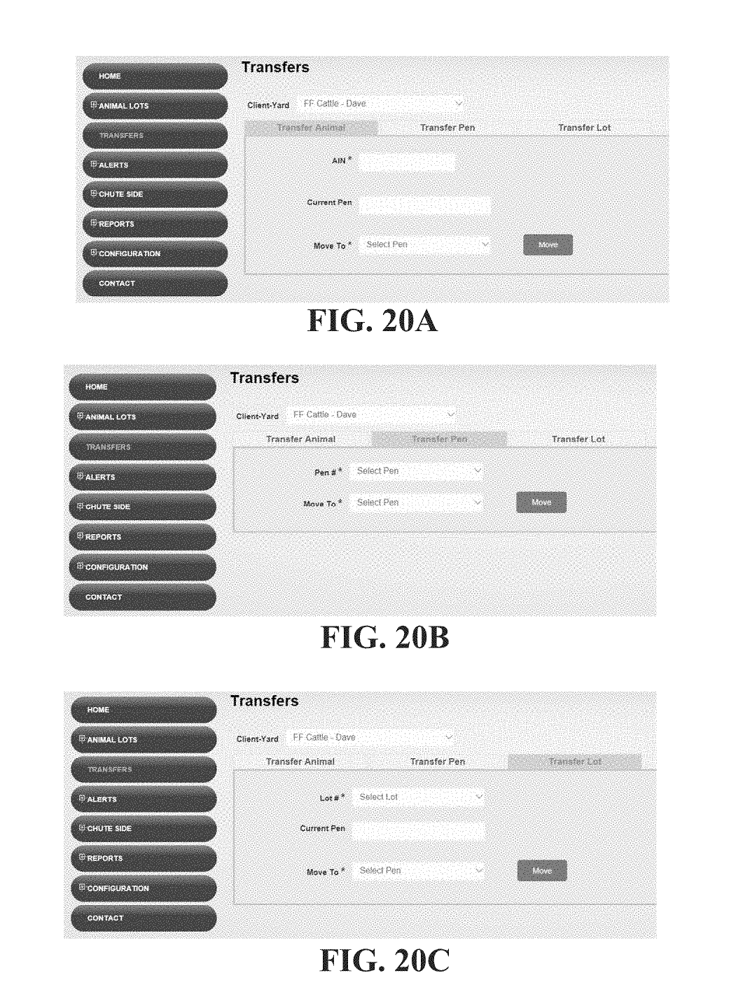

FIG. 15 illustrates an example non-confirming list that data representation generator 392 may generate if no animals satisfy the non-confirming criteria (which may be predefined by a user). As shown, the list is empty. FIG. 15 also shows other tabs that contain other information described by their titles. FIG. 16 illustrates an example “All animals” list that data representation generator 392 may generate for a user who makes one or more selections to see all of the animals in a given Pen, Lot, Yard, Operation, or accessible to system 1000 generally. FIG. 17 illustrates an example of information that data representation generator 392 may provide under the “Follow-Up” tab to a user in conjunction with the treatment selector 360 of computing platform 300. As shown, the table indicates the number of animals that are eligible for revaccination as of a certain date. FIG. 18 illustrates a lot creation feature of the GUI that may enable a user to define or assign what animals, hardware, etc. are associated with what lots of the operation. FIG. 18 illustrates a tag pairing feature of the GUI that enables a user to manually input information relevant to a particular animal and pair the HMD's (i.e., the “tags”) with given animals as they deem appropriate, among other information. FIG. 20 illustrates a Transfer Animal feature that allows a user to reassign a given animal from one pen to another from the perspective of the system 1000.

FIG. 20A illustrates a Transfer Animal feature that allows a user to reassign a given animal from one pen to another from the perspective of the system 1000. FIG. 20B illustrates a Transfer Pen feature that allows a user to make a pen reassignment from the perspective of the system 1000. FIG. 20B illustrates a Transfer Pen feature that allows a user to make a pen reassignment from the perspective of the system 1000. FIG. 20C illustrates a Transfer Lot feature that allows a user to reassign a lot designation from one pen to another from the perspective of the system 1000.

FIG. 21 illustrates an example AMM App welcome screen (which may be shown after a user logs in with their credentials) including one or more status indications 412, example menu items including menu button 414 associated with Today's Pull List, menu button 416 for Today's Non-Conforming List, among other pieces of information. Also shown in FIG. 21 are various example peripherals, including but not limited to: peripheral 450 which may be any peripheral device that may be operably connected with and transmit information to client computing device 400; peripherals 418 and 420 which may include a camera and light source respectively; peripheral 418 which may include a speaker and/or a microphone, and peripheral 428 which may include a biometric fingerprint scanner (which may be operable for authenticating a user in some embodiments).

FIG. 22A illustrates an example Pull List as described hereinabove. FIG. 22B illustrates an example empty Pull List as described hereinabove. FIG. 23 illustrates an example Non-Conforming List as described hereinabove.

FIG. 24 illustrates a graphical depiction of example user categories—general manager, supervisor, and handler—and their caregiving responsibilities. For example, General Manager oversees Supervisor A, Supervisor B, and Supervisor C, who in turn oversee multiple handlers and are responsible for certain animals in an operation. The General Manager's responsibilities extend to all the Sheep, Cattle and Pigs in the operation, and all the other caregivers under his/her supervision. Supervisor A's responsibilities extend to all the Sheep, and all the other caregivers under his/her supervision (e.g., Handler 1, Handler 2, Handler 3) who handle subsets of the Sheep population on the farm, and so on and so forth for Supervisor B and Supervisor C.

The present disclosure teaches various operations, computations, derivations and manipulations of information about a given animal, whether originating from the HMD 100, entered manually by a user, or obtained from an external resource 600. Though specific examples and arrangements are discussed in this disclosure for clarity of description, it should be understood that other variations and arrangements may be implemented. For instance, any of the elements of the system may include computing resources such as a memory, a processing engine, a storage medium, and the like. As such, the particular example arrangements discussed herein should not be read to limit the disclosure to the particular form, and it should be understood that the various operations, computations, derivations and manipulations of information disclosed herein may be performed by the computing resources of any one or more of the elements of the system (e.g., HMDs 100, Receivers 200, Computing Platforms 300, client computing devices 400) or any sub-elements or components of those elements.

Referring to FIGS. 1-24 collectively, although these illustrate example embodiments with components, elements, circuits and other items partitioned in the depicted manner, it will be appreciated by one of ordinary skill in the art that various components and circuits of the system 1000, HMD 100, and subsystems described herein may be implemented utilizing any form of hardware, software, or a combination thereof. For example, one or more processors, controllers, ASICs, PLAs, PALs, CPLDs, FPGAs, logical components, software routines or other mechanisms, including associated memory, might be used to implement one or more components or circuits in embodiments of the system 1000 or HMD 100, or other components of the present disclosure. In embodiments, the various components and circuits described herein might be implemented as discrete components or the functions and features described can be shared in part or in total among two or more components. In other words, as would be apparent to one of ordinary skill in the art after reading this description, the various features and functionality described herein may be implemented in any given application and can be implemented in one or more separate or shared components in various combinations and permutations. Even though various features or elements of functionality may be individually described or claimed as separate components, in various embodiments these features and functionality can be shared among one or more common software and hardware elements, and such description shall not require or imply that separate hardware or software components are used to implement such features or functionality.

HMDs or other system elements of the present disclosure might include, for example, one or more processors, controllers, control modules, or other processing devices. Such might be provided by general-purpose or special-purpose processing engines such as, for example, a microprocessor, controller, or other control logic.

HMDs or other system elements of the present disclosure might include one or more memory modules, simply referred to herein as memory. For example, memory might include random access memory (RAM) or other dynamic memory which might be used for storing information and instructions to be executed by a processing engine of a HMD or other system elements. Memory might also be used for storing temporary variables or other intermediate information during execution of instructions to be executed by the HMDs' or other system elements' processing engine. Memory might likewise include a read only memory (“ROM”) or other static storage device coupled to a bus for storing static information and instructions for an associated processor.

It will be understood by those skilled in the art that the HMDs or other system elements of the present disclosure might include one or more various forms of information storage mechanism, which might include, for example, a media drive and a storage unit interface. The media drive might include a drive or other mechanism to support fixed or removable storage media. For example, a hard disk drive, a solid-state drive, a magnetic tape drive, an optical disk drive, a CD, DVD, or Blu-ray drive (R or RW), or other removable or fixed media drive might be provided. Accordingly, storage media might include, for example, a hard disk, a solid-state drive, magnetic tape, cartridge, optical disk, a CD, DVD, Blu-ray or other fixed or removable medium that is read by, written to or accessed by media drive. As these examples illustrate, the storage media can include a computer usable storage medium having stored therein computer software or data.

In alternative embodiments, information storage mechanisms that may be implemented in one or more embodiments of the present disclosure might include other similar instrumentalities for allowing computer programs or other instructions or data to be loaded into one or more computing components of HMDs or other system elements. Such instrumentalities might include, for example, a fixed or removable storage unit and an interface. Examples of such storage units and interfaces can include a program cartridge and cartridge interface, a removable memory (for example, a flash memory or other removable memory module) and memory slot, a PCMCIA slot and card, and other fixed or removable storage units and interfaces that allow software and data to be transferred from the storage unit to the HMD or other system elements (e.g., to a memory of the HMD).

As described herein, and as one of ordinary skill in the art will appreciate, HMDs or other system elements of the present disclosure might include a communications interface. Such communications interfaces might be used to allow software and data to be transferred between the HMDs or other system elements and external devices or resources. Additional nonlimiting examples of communications interfaces might include a modem or softmodem, a network interface (such as an Ethernet, network interface card, WiMedia, IEEE 802.XX or other interface), a communications port (such as for example, a USB port, IR port, RF port, RS232 port Bluetooth® interface, or other port), or other communications interfaces. Software and data transferred via a communications interface might typically be carried on signals, which can be electronic, electromagnetic (which includes optical) or other signals capable of being exchanged by a given communications interface. These signals might be provided to the communications interface via a channel. This channel might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium,” “machine readable medium” and “computer usable medium” are used to generally refer to transitory or non-transitory media such as, for example, memory, storage unit, media, and channel discussed above. These and other various forms of computer program media, computer readable media, or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable HMDs or other system elements to perform features or functions of the present application as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module and component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the terms “module” or “component” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An intelligent health monitoring system comprising:
a processor;
a memory;
a communications interface that receives data from a remote intelligent health monitoring device;
a non-transitory computer readable medium storing machine readable instructions that, when executed by the processor, cause the intelligent health monitoring engine to:
acquire temperature data associated with an animal based on one or more temperature measures obtained by remote intelligent health monitoring device;
determine whether the temperature data acquired satisfies a health condition criteria;
identify a treatment modality to be delivered to the animal to treat a health condition if a health condition criteria has been satisfied;
identify a caregiver for the animal;
generate a data representation for display on a remote computing device running an animal management application having a graphical user interface accessible to the identified caregiver;
providing the data representation for display on the remote computing device running the animal management application.

2. The intelligent health monitoring engine of claim 1, wherein the data representation comprises a pull list identifying animals that need to be treated for a health condition.

3. The intelligent health monitoring engine of claim 1, wherein the data representation comprises a non-conforming list identifying animals that were previously listed on a pull list but were not pulled for treatment of the health condition.

4. The intelligent health monitoring engine of claim 1, wherein the data representation comprises a consecutive missed list identifying animals that were previously listed on a pull list and previously listed on a non-conforming list but were still not pulled for treatment of the health condition.

5. The intelligent health monitoring engine of claim 1, wherein determining whether temperature data satisfies a health condition criteria is based upon one or more of the ambient temperature near the animal's location, the length of time during which the temperature data satisfied the health condition criteria.

6. The intelligent health monitoring engine of claim 1, wherein identifying a treatment modality to be delivered to the animal to treat a health condition is based on one or more of: the animal's breed, age, size, treatment history, travel history, altitude information, and known allergies.

7. The intelligent health monitoring engine of claim 1, wherein identifying a treatment modality to be delivered to the animal to treat a health condition is based on one or more of: the treatment's availability, effectiveness, and cost.

8. The intelligent health monitoring engine of claim 1, wherein identifying a treatment modality to be delivered to the animal to treat a health condition comprises selecting among a plurality of medications to deliver to the animal.

9. The intelligent health monitoring engine of claim 1, wherein providing treatment modality information to the caregiver comprises: generating one or more of a textual, graphical, audio or video message accessible through a graphical user interface of the animal management application.

10. The intelligent health monitoring engine of claim 1, wherein treatment modality information comprises one or more of: a medication type, availability, dosage, dosage schedule, and delivery site.

11. An intelligent health monitoring engine comprising:
a processor;
a memory;
a communications interface to receive data from a remote intelligent health monitoring device;
a non-transitory computer readable medium storing machine readable instructions that, when executed by the processor, cause the intelligent health monitoring engine to:
acquire temperature data associated with an animal based on one or more temperature measures obtained by remote intelligent health monitoring device;
determine whether temperature data satisfies a health condition criteria;
identify a treatment modality to be delivered to the animal to treat a health condition if a health condition criteria has been satisfied;
identify a caregiver for the animal;
provide treatment modality information to the caregiver by generating a data representation for display on a remote computing device running an animal management application having a graphical user interface accessible to the identified caregiver;
providing the data representation for display on the remote computing device running the animal management application.
wherein the intelligent health monitoring device comprises: a housing comprising a casing member releasably couplable with a base member, wherein the casing member and the base member create an internal cavity when in a coupled configuration;
an environment resistant seal disposed between the casing member and the base member, wherein the environment resistant seal is held between the casing member and the base member when the casing member and the base member are in the coupled configuration;
a stud coupled to a side of the base member, the stud configured to pierce biological tissue;
a circuit configured to support electrical connections, the circuit supporting electrical connections between:
a processing engine;
a memory;
a transmitter; and
a power source;
wherein the processing engine, memory, transmitter circuit, and power source are operatively coupled together and held within the internal cavity;
a conductor operatively coupled to the circuit and a heat sensor, a portion of the conductor disposed within a flexible cord passing through an opening in the housing and extending outside the internal cavity to at least a portion of the heat sensor.

12. The intelligent health monitoring engine of claim 11, wherein the data representation comprises a pull list identifying animals that need to be treated for a health condition.

13. The intelligent health monitoring engine of claim 11, wherein the data representation comprises a non-conforming list identifying animals that were previously listed on a pull list but were not pulled for treatment of the health condition.

14. The intelligent health monitoring engine of claim 11, wherein the data representation comprises a consecutive missed list identifying animals that were previously listed on a pull list and previously listed on a non-conforming list but were still not pulled for treatment of the health condition.

15. The intelligent health monitoring engine of claim 11, wherein identifying a treatment modality to be delivered to the animal to treat a health condition is based on one or more of: the animal's breed, age, size, treatment history, and known allergies.

16. The intelligent health monitoring engine of claim 11, wherein identifying a treatment modality to be delivered to the animal to treat a health condition is based on one or more of: the treatment's availability, effectiveness, and cost.

17. The intelligent health monitoring engine of claim 11, wherein identifying a treatment modality to be delivered to the animal to treat a health condition comprises selecting among a plurality of medications to deliver to the animal.

18. The intelligent health monitoring engine of claim 11, wherein providing treatment modality information to the caregiver comprises: generating one or more of a text message, an email, or a phone call describing the treatment modality information.

19. The intelligent health monitoring engine of claim 11, wherein treatment modality information comprises one or more of: a medication type, availability, dosage, and delivery site.

20. An intelligent health monitoring engine comprising:
a processor;
a memory;
a communications interface to receive data from a remote intelligent health monitoring device;
a non-transitory computer readable medium storing machine readable instructions that, when executed by the processor, cause the intelligent health monitoring engine to:
acquire physical parameter data associated with an animal based on one or more physical parameter measures obtained by remote intelligent health monitoring device;
determine whether the physical parameter data satisfies a health condition criteria;
identify a treatment modality to be delivered to the animal to treat a health condition if a health condition criteria has been satisfied;
identify a caregiver for the animal;
provide treatment modality information to the caregiver;
generate a data representation for display on a remote computing device running an animal management application having a graphical user interface accessible to the identified caregiver, the data representation including identification information for the animal to which the treatment modality is to be delivered;
providing the data representation for display on the remote computing device running the animal management application.

21. The intelligent health monitoring engine of claim 11, wherein the remote intelligent health monitoring device is attached to the ear of the animal, and the physical parameter data is based on heat sensed within the ear canal of the animal.
